(19) United States
(12) Patent Application Publication
(10) Pub. No.: US 2024/0024540 A1
(43) Pub. Date: Jan. 25, 2024

(54) Drug-releasing catheter extender-lengthener and catheter extension tube

(71) Applicant: Novaflux Inc., Princeton, NJ (US)

(72) Inventors: Stephen Richard Ash, Lafayette, IN (US); Mohamed Emam Labib, West Palm Beach, FL (US); Lucas Lawrence Franz, Mount Laurel, NJ (US)

(21) Appl. No.: 18/198,755

(22) Filed: May 17, 2023

Related U.S. Application Data
(63) Continuation-in-part of application No. 17/986,604, filed on Nov. 14, 2022.
(60) Provisional application No. 63/350,621, filed on Jun. 9, 2022.
(60) Provisional application No. 63/390,166, filed on Jul. 18, 2022.
(60) Provisional application No. 63/278,595, filed on Nov. 12, 2021.

(51) Int. Cl. A61L 29/16; A61M 25/00

(52) U.S. Cl. CPC A61L 29/16; A61M 25/00; A61L 2300/406; A61M 2209/04

(57) ABSTRACT

In an embodiment, there is provided a catheter extender-lengthener comprising end connectors and tubing that comprises a polymer and a substance such as an antibiotic that is safe for exposure to bodily fluids. The catheter extender-lengthener is suitable to connect to a catheter extension set. Antibiotic can be eluted to fluid on both sides of a clamp and can diffuse toward the patient's body. Similar tubing can be used in a catheter extension set and optionally in a cap. The catheter extender-lengthener can be added on to existing catheter assemblies. Drugs can be combined at high concentration with polymer, and the device can elute effective amounts of minocycline and rifampin for more than a month. Use of the devices can provide adjustable antibiotic or dose and can be adjusted in response to a patient's condition. This can lessen the possibility of Catheter Related Blood Stream Infection.

Priority is claimed, to the extent appropriate, to U.S. Application Ser. No. 63/350,621 filed Jun. 9, 2022 (docket number 14605.0040USP1) and U.S. Application Ser. No. 63/390,166 filed Jul. 18, 2022 (docket number 14605.0040USP2). This patent application is a Continuation-in-Part of U.S. application Ser. No. 17/986,604 filed Nov. 14, 2022 (docket number 14605.0037USU1), which in turn claims priority, to the extent appropriate, to U.S. Application Ser. No. 63/278,595, filed Nov. 12, 2021 (docket number 14605.0037USP1). All of these applications are incorporated herein by reference in their entireties.

### BACKGROUND OF THE INVENTION

Catheters extend between regions that are internal to the body, and regions that are external to the body and may be non-sterile. Transdermal catheters cross the skin boundary. Catheters are potential routes for introduction of bacteria, fungi or other pathogens inside the body. Currently, many catheters (such as central venous catheters for infusion or dialysis) have to be replaced periodically simply because of the growth of biofilm within them and subsequent bacterial infection in the blood (bacteremia, i.e., Catheter Related Blood Stream Infections (CRBSI)). Over a period of use, biofilm grows from the hub or similar external part of the catheter through the catheter lumen towards the tip of the catheter in the patient's body. This can occur anywhere in body where the catheter is used.

Therefore, efforts have been made to kill bacteria or discourage bacterial or biofilm growth within catheters. Antibacterial substances of interest include disinfectants such as chlorhexidine, and antibiotics. It can be noted, however, that if disinfectants are used, the method of use must be such that there is no possibility of the disinfectants mixing with internal bodily fluids such as blood. Disinfectants and antiseptics kill bacteria by wide-ranging chemical effects which can kill or damage living cells in the body, especially in the blood, where they may cause systemic toxicity. One method of administration of disinfectant has been as a disinfectant-impregnated rod in a catheter cap, which may be part of a closure cap that closes the outer end of the catheter extension set. This may comprise a rod dimensioned so as to partially and temporarily occupy some of the interior space and which may have a material composition or a coating that can elute the desired disinfectant. The cap typically is able to be connected to the catheter or hub and able to be removed from the catheter or hub as desired. Such a cap must always be separated from bodily fluids such as by a clamp on the tubing. Some early patents in the field disclose the antimicrobial as being an iodine substance or other disinfectant compounds.

Central venous catheters used for infusion or withdrawing blood have hubs which allow connection of Luer-lock tips of syringes and various medical tubings. The catheter (i.e., the part which enters the patient's body) does not connect directly to the hub, but rather the catheter connects to clear, flexible short tubing sections called an “extension set” or extension tubing, which remains outside the patient's body. The catheter portion is permanently assembled to or permanently connected to the extension set before the product is delivered to the user or hospital. The catheter may contain several lumens leading to the central vein. If so, there is provided a separate extension tubing for each lumen. The “extension set” typically is suitable such that its tubing can be compressed or clamped so that fluid communication is shut off. During use, the clamp is closed before the cap of the hub is removed, to prevent exit of blood from or influx of air into the catheter lumen. After a syringe or medical tubing is attached to the connector or hub at the end of the extension tubing, the clamp can be opened to allow passage of various fluids to and from and through the catheter lumen.

ClearGuard HD® (ICU Medical, San Clemente, CA) is a known catheter cap that releases chlorhexidine from a rod fixed to or integral with a cap. Specifically, the chlorhexidine is released from a coating on the rod. The ClearGuard product is effective to reduce infection by about 75%, which is a worthwhile improvement. Chlorhexidine is effective against bacteria but, because of its toxicity, it must not be allowed to enter the patient's body. The clamp on the extension set is placed along the length of the tube before the cap is placed on the catheter hub. Chlorhexidine and most other non-specific antiseptics/disinfectants are toxic to blood, so that is why the rod and cap are always used conjunction with a clamp on the tube. Therefore, chlorhexidine or similar substance can kill bacteria in the region near the cap but cannot get past the clamp and into bloodstream, due to the closed clamp. If a device is used with a disinfectant such as chlorhexidine, there must always be a clamp or similar feature between that region and the patient so as to isolate and keep the region where the disinfectant is being emitted away from bodily fluids.

It would be inappropriate or risky to put chlorhexidine in the material of which the catheter extension tube itself is made, or the tubing of which the catheter extension set is made, because in that situation, the chlorhexidine would diffuse from the portion of the extension tube which is between the clamp and the catheter lumen, and from there would diffuse down the catheter lumen and into the blood stream causing systemic toxicity. There is no FDA approval for the use of chlorhexidine in such a situation. So, incorporating a substance into the material of which the wall of the catheter extension tube is made only is practical if the substance being eluted is an antibiotic suitable for use internal to the body, rather than an antiseptic or disinfectant.

In the known commercial product ClearGuard product, the cap comprises a rod extending from the cap into the tubing, and the rod delivers chlorhexidine to an isolated volume of liquid, which typically is isolated by a clamp on tubing. Isolation is necessary because chlorhexidine being in fluid communication with the blood stream is unacceptable because chlorhexidine has toxicity and will significantly damage red blood cells and promote clotting of blood and cause systemic toxicity. Chlorhexidine has FDA approval only for uses in which it can be guaranteed that none of the chlorhexidine would ever reach the patient's blood stream.

During use of a catheter, it is typical to clamp the catheter tubing while disconnecting any item from the Luer lock hub, in order to block entry of air and prevent exit of blood or other fluids. Typically the clamp is a pinch clamp. Clamps on catheter extension sets are an integral part of extension tubings and if a clamp is not present it is not possible to use the catheter port safely for drawing blood or infusing fluids. The clamp also serves as backup closure to the cap in case the cap falls off or is lost. If a clamp is present and is being used with using a rod such as the ClearGuard product, the rod is dimensioned to be sufficiently short so that clamping can occur at a location on the extension tube where the clamp does not have to clamp around the rod. If the clamp is a pinch clamp and a portion of the rod were underneath the pinch clamp when it was compressed, the pinch clamp would not be very effective in closing off the catheter extension tube from chlorhexidine release, and this also would increase the risk of damage to the tubing in the region of the pinch clamp.

Therefore, in current practice, the clamping device would typically clamp the tubing at a location at which the rod is not present so that the tubing can fully collapse against itself to block the passage of fluid.

It also is known to introduce or deposit an antibacterial or antibiotic substance into the wall of tubing by solvent deposition (infusion), but the amount of the antibacterial or antibiotic substance that can be so deposited is limited.

Another approach known in catheter technology is a coating material for providing an antibacterial material available to the fluid inside the catheter. However, such material is only a coating rather than being distributed throughout the catheter material. Such coating typically only lasts for a few days, and has not achieved desirably long release of antibacterial substance.

As an alternative to the use of chlorhexidine or similar antiseptics, it would be desirable to provide some antibacterial or antimicrobial such as an antibiotic. Eluting antibiotic for a week or for a plurality of weeks would help to prevent catheter related blood stream infections.

### SUMMARY OF THE INVENTION

Embodiments of the present invention offer the possibility of reducing infection to an even greater extent than known technology, while also simplifying procedures and protocols during use.

In an embodiment of the present invention, there is provided a catheter extender-lengthener comprising: a first connector, the first connector being suitable to engage with a corresponding connector of a catheter or a catheter extension set; at least one tube proceeding from the first connector, the tube having a tube wall; and a second connector connected proceeding from the tube, wherein the tube wall comprises a material that comprises a polymer and at least one antibiotic.

In an embodiment of the invention, there is provided a catheter extender-lengthener for use with a catheter, the catheter extender-lengthener comprising: a first connector, the first connector being suitable to engage with a corresponding connector of a catheter or a catheter extension set; a catheter extension tube proceeding from the first connector, the catheter extension tube having a catheter extension tube wall containing at least one antibiotic substance that is safe for exposure to a patient's bodily fluids; and a second connector connected proceeding from the catheter extension tube; the first connector, the catheter extension tube and the second connector defining a lumen extending therethrough, further comprising a clamp disposed on the catheter extension tube having at least one permitted location on the catheter extension tube, wherein when the clamp is in a permitted location on the catheter extension tube, fluid inside the lumen on one side of the clamp is exposed to the polymer and the at least one antibiotic substance, and fluid inside the lumen on an opposite side of the clamp is exposed to the polymer and the at least one antibiotic substance.

In an embodiment of the invention, there is provided a catheter assembly of a catheter and a catheter extension set, the catheter assembly comprising: the catheter, the catheter being suitable to extend into a patient's body; a junction between the catheter and the catheter extension set; and the catheter extension set, the catheter extension set comprising: at least one catheter extension tube proceeding from the junction, the catheter extension tube having a catheter extension tube wall; and an end connector proceeding from the catheter extension tube, wherein the catheter, the junction, the catheter extension tube and the end connector define a lumen extending therethrough, wherein the catheter extension tube wall comprises a polymer and at least one antibiotic or a combination of antibiotics, and wherein the catheter extension tube wall has a composition different from a composition of the catheter.

In an embodiment of the invention, there is provided a cap for connecting onto a connector, the cap comprising: an engagement means for engaging with the connector and forming a fluid-tight seal with the connector; and a projection connected to the cap, wherein the projection comprises a polymer and at least one antibiotic that is safe for exposure to a patient's bodily fluids.

In an embodiment of the invention, there is provided a method of limiting infection in a patient, the method comprising: implanting a catheter in the patient, the catheter being part of a catheter assembly that comprises the catheter and a catheter extension set, wherein either the catheter or the catheter extension set or both comprises tubing whose walls contain an antibiotic; monitoring bodily indicators of the patient; and based upon the bodily indicators, attaching a first catheter extender-lengthener to an end of the catheter assembly, wherein the catheter extender-lengthener comprises a polymer and at least one antibiotic.

### SUMMARY OF THE ILLUSTRATIONS

Embodiments of the invention are further described, but are in no way limited, by the following illustrations.

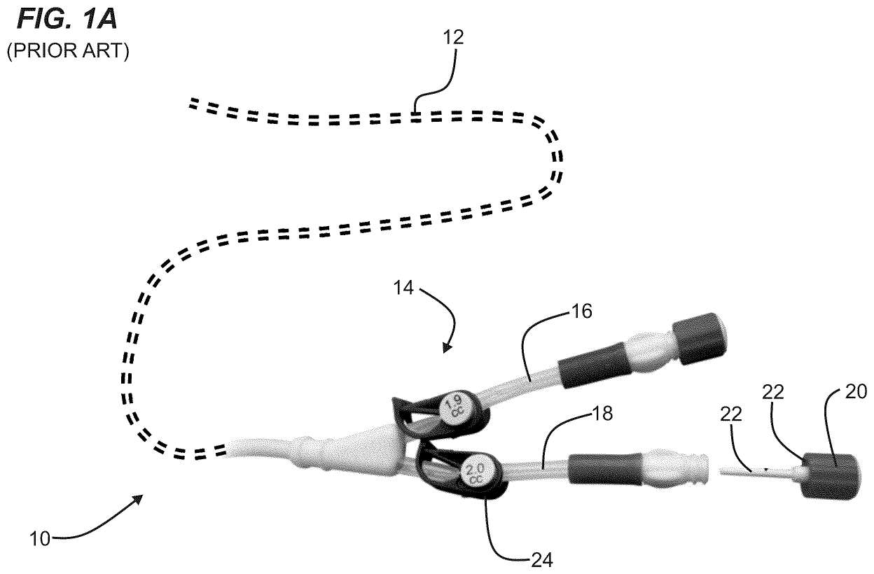

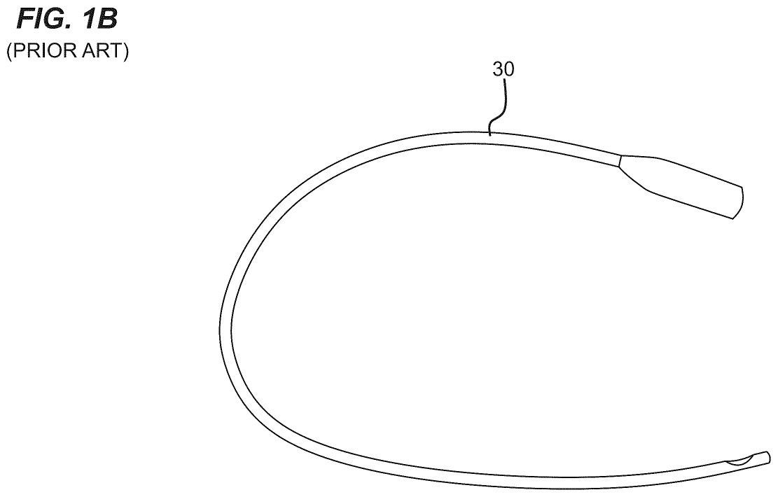

FIG. 1A shows a prior art catheter assembly comprising a catheter and a catheter extension set, in combination with a conventional cap and rod that contains an antiseptic that is released to kill bacteria in the hub and outer part of the extension tube.

FIG. 1B shows a prior art catheter that releases antibiotic from the wall of the tube.

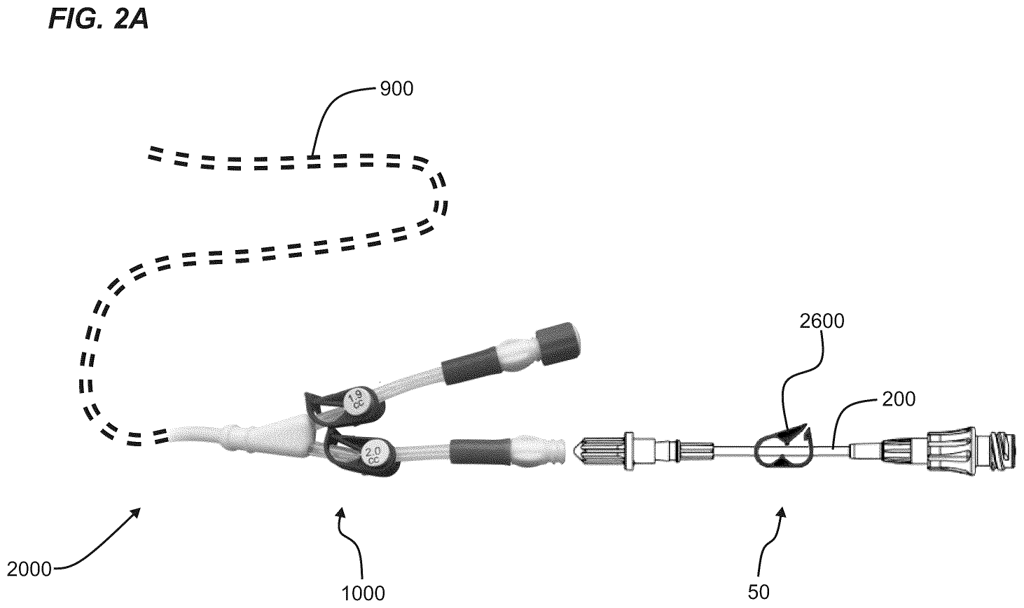

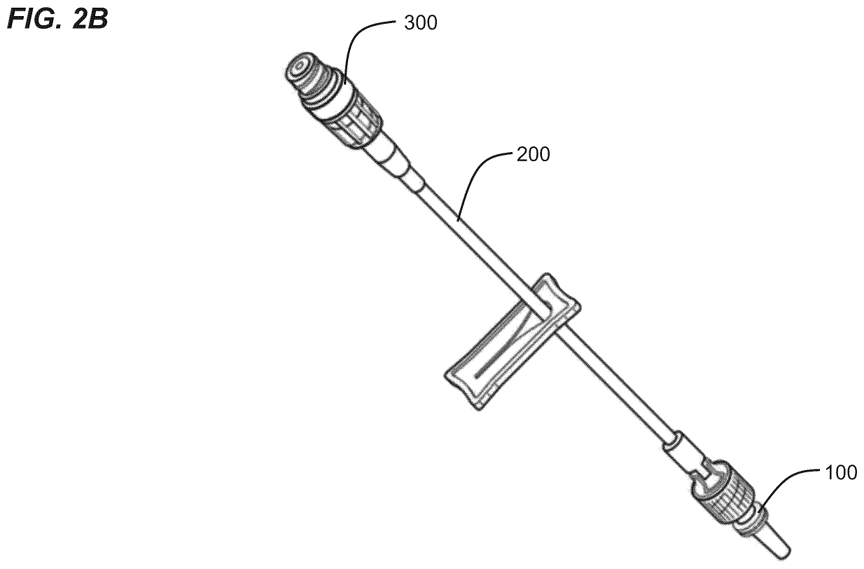

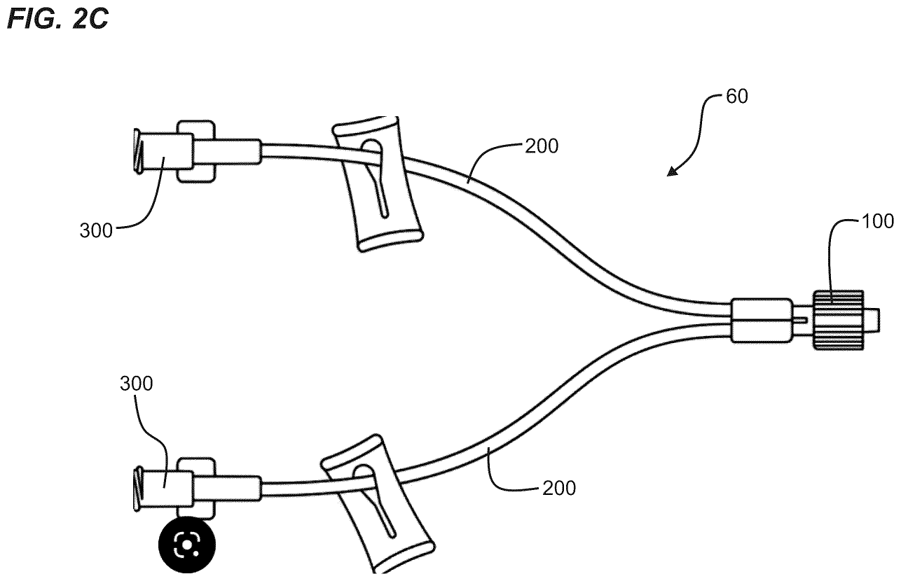

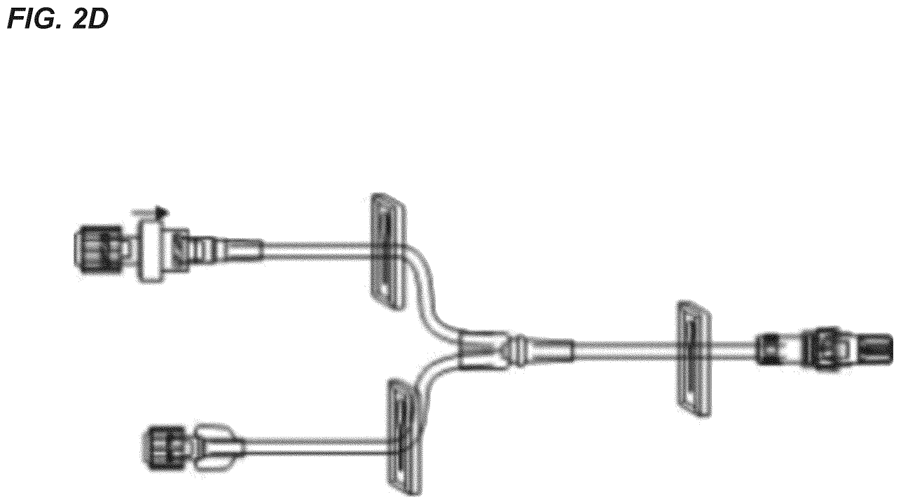

FIG. 2A shows a catheter extender-lengthener of an embodiment of the invention, which may comprise a polymer that elutes antibiotic. Such a catheter extender-lengthener is not manufactured with the rest of the catheter, but rather is a separate piece that can be connected to a catheter assembly as an add-on device. A cooperating catheter assembly is also shown in FIG. 2A.

FIG. 2B shows another view of the catheter extender-lengthener of FIG. 2A, in isolation.

FIG. 2C shows a catheter extender-lengthener similar to the one of FIG. 2B, but containing a branching.

FIG. 2D shows a catheter extender-lengthener similar to the one of FIG. 2C, but containing another form of branching.

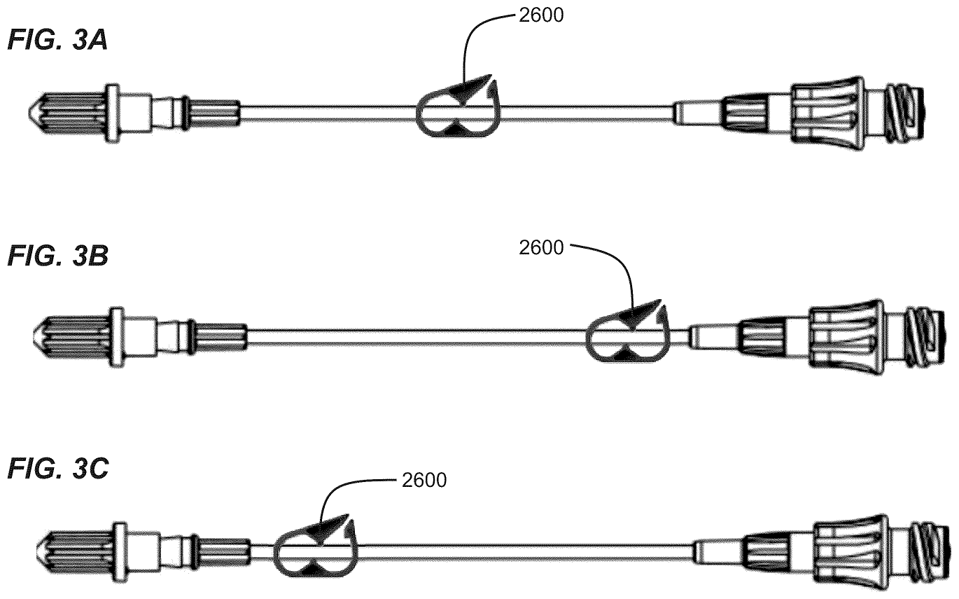

FIGS. 3A-3C show a catheter extender-lengthener with various locations of a clamp.

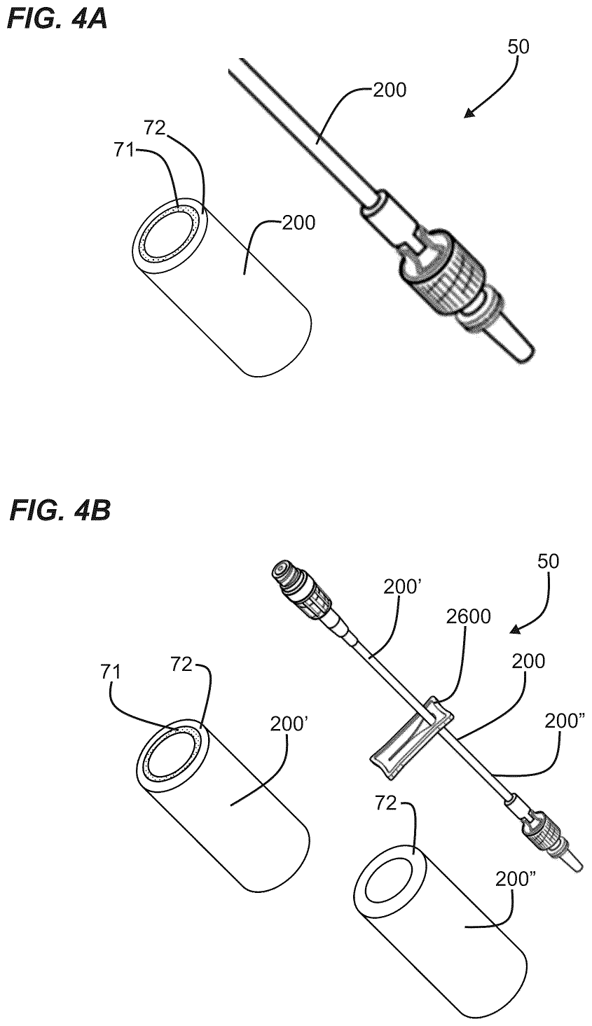

FIG. 4A shows a catheter extender-lengthener in which there are different compositions at different radial locations within the cross-section of the catheter extension tube.

FIG. 4B shows a catheter extender-lengthener in which there are different compositions at different places along the length of the catheter extension tube.

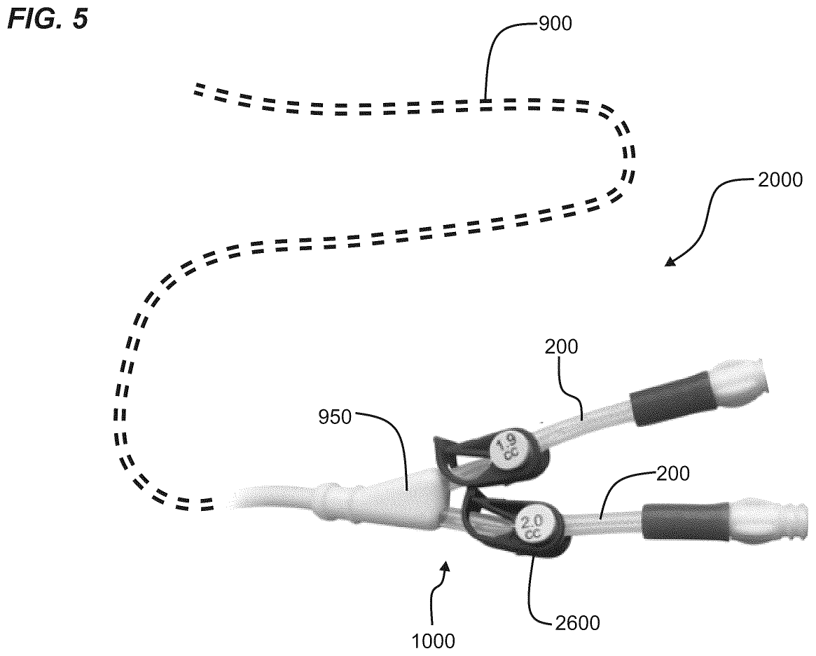

FIG. 5 shows a catheter assembly comprising a catheter and a catheter extension set, with antibiotic-eluting polymer in the catheter extension set.

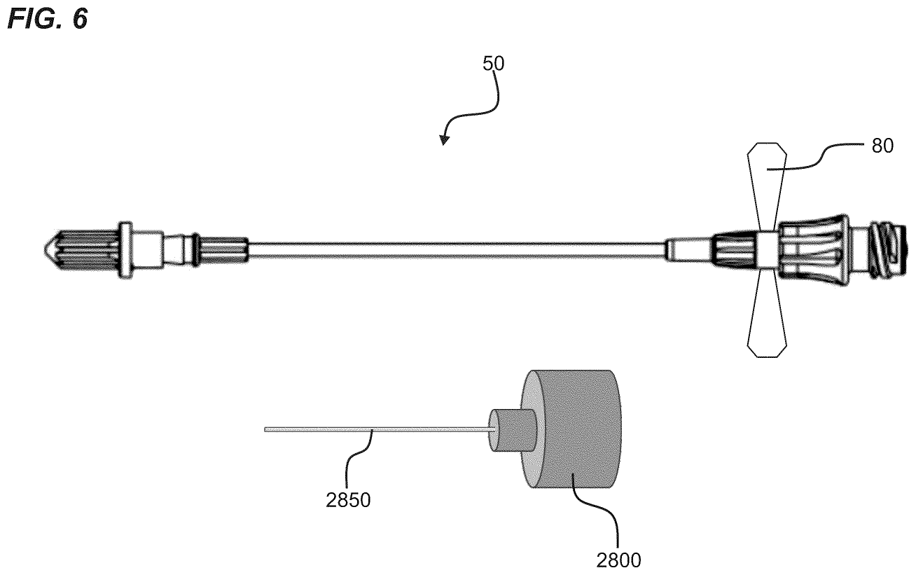

FIG. 6 shows a cap having a projection comprising antibiotic-eluting polymer.

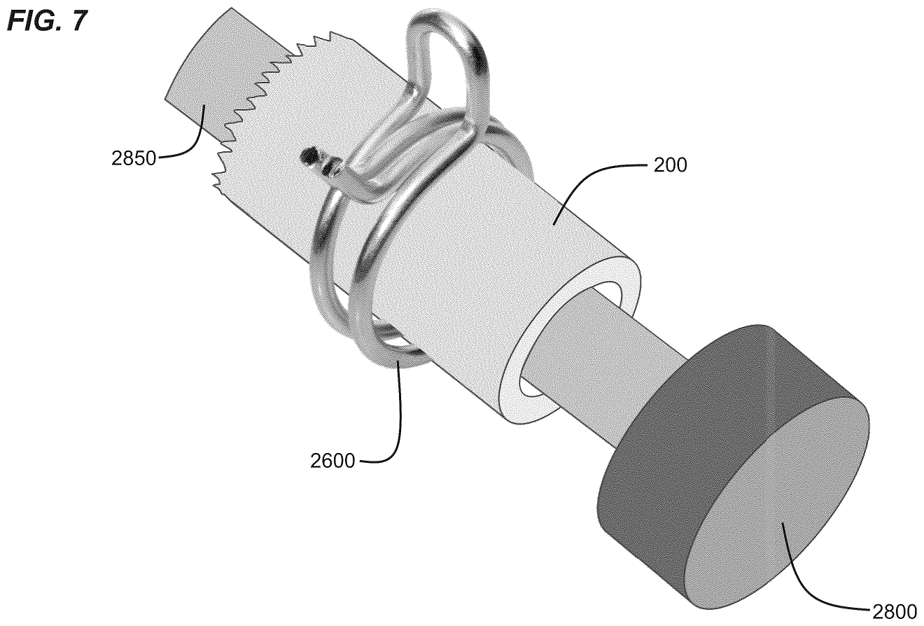

FIG. 7 shows such a cap in combination with tubing and a clamp that squeezes the tubing uniformly around the projection from the cap.

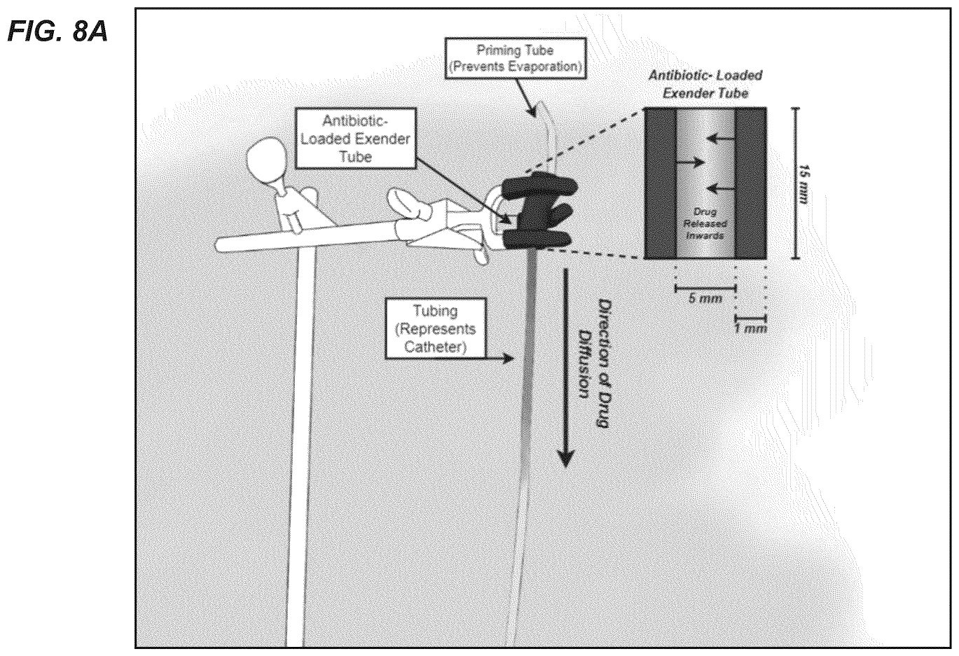

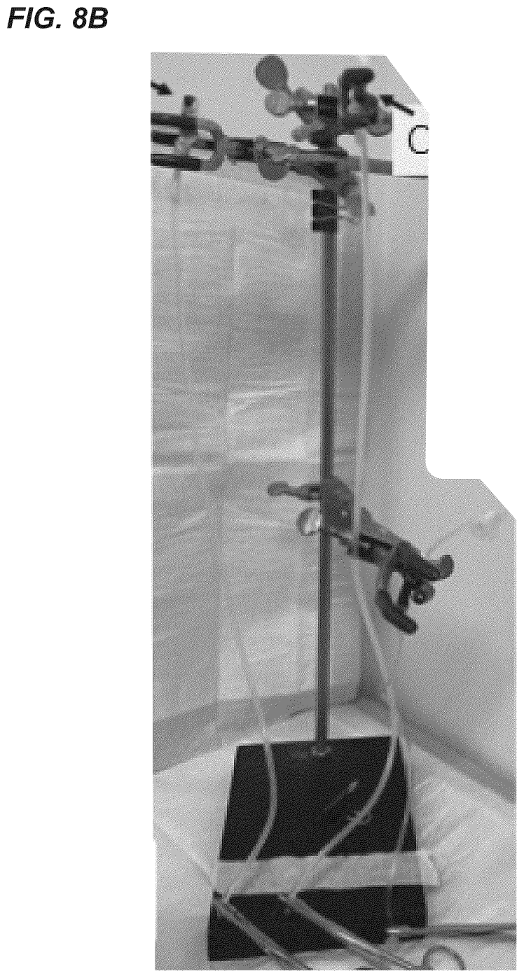

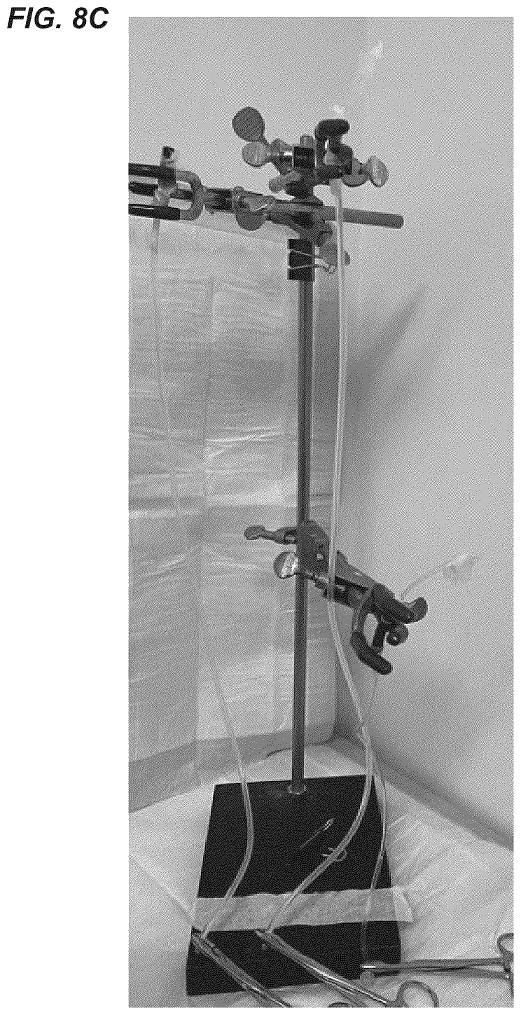

FIGS. 8A-8C show experimental arrangements for measuring release of drug from tube wall to fluid in the lumen.

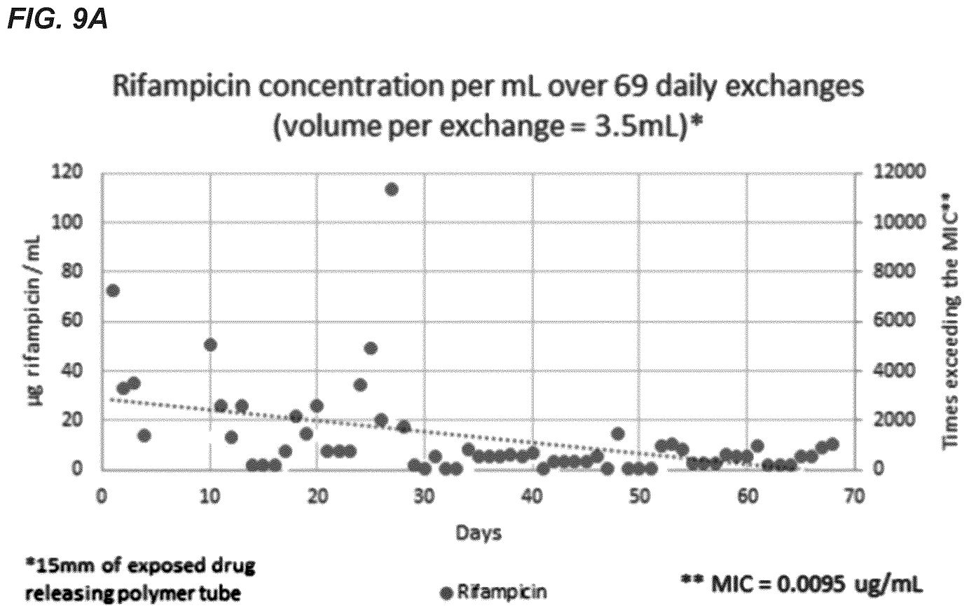

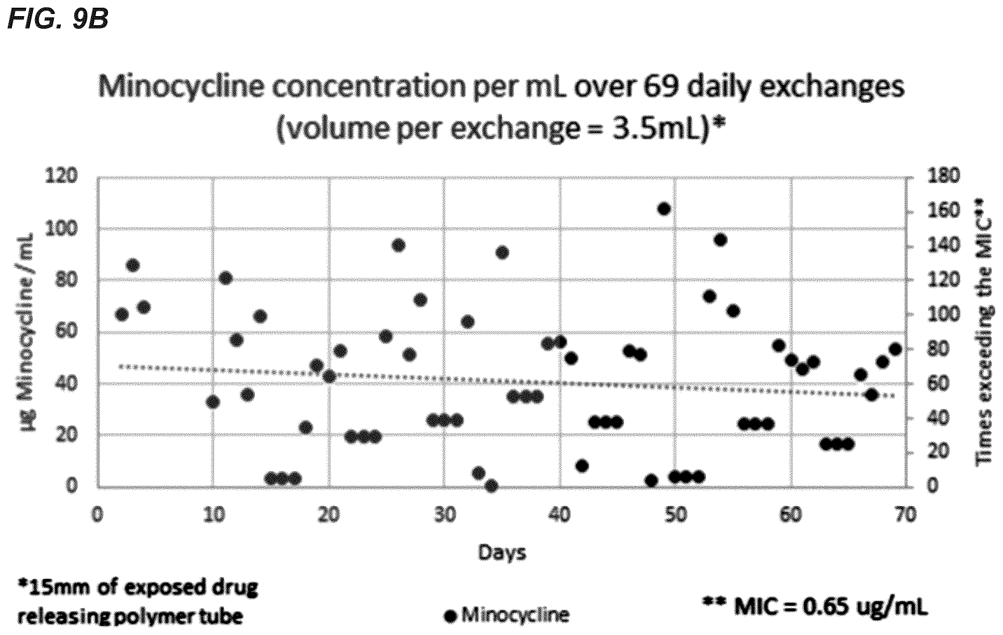

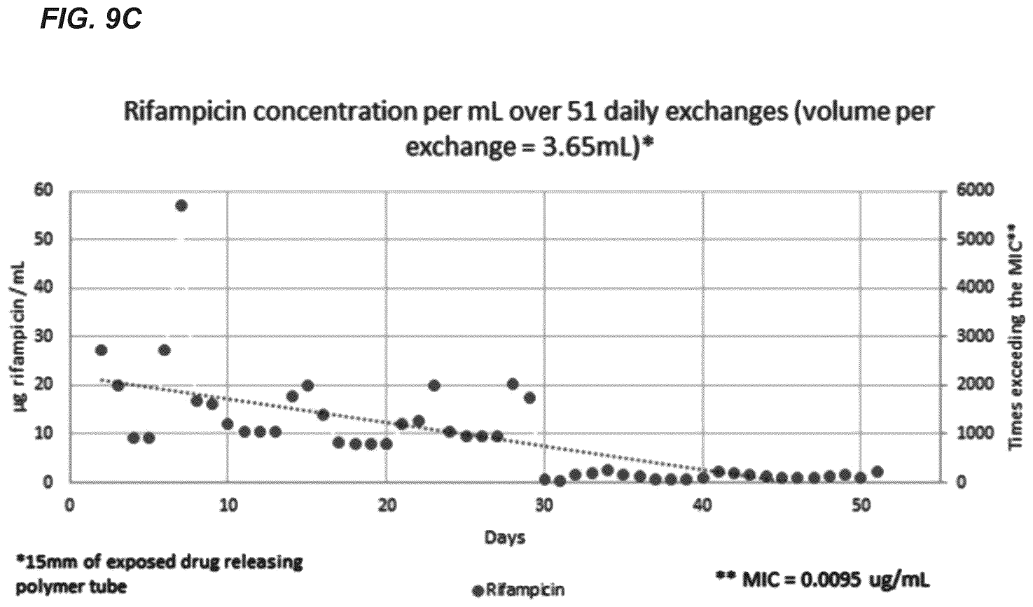

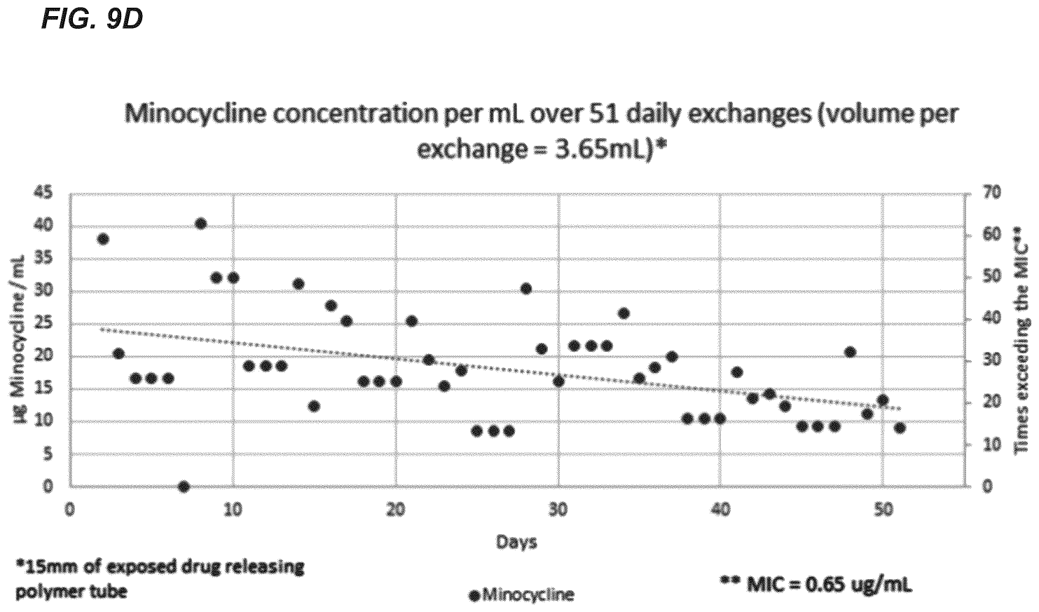

FIGS. 9A-9D are ex vivo experimental results showing release of drug during given intervals, for a geometry in which the drug-releasing polymer was in the form of a tube and the fluid was contained in the lumen of the tube.

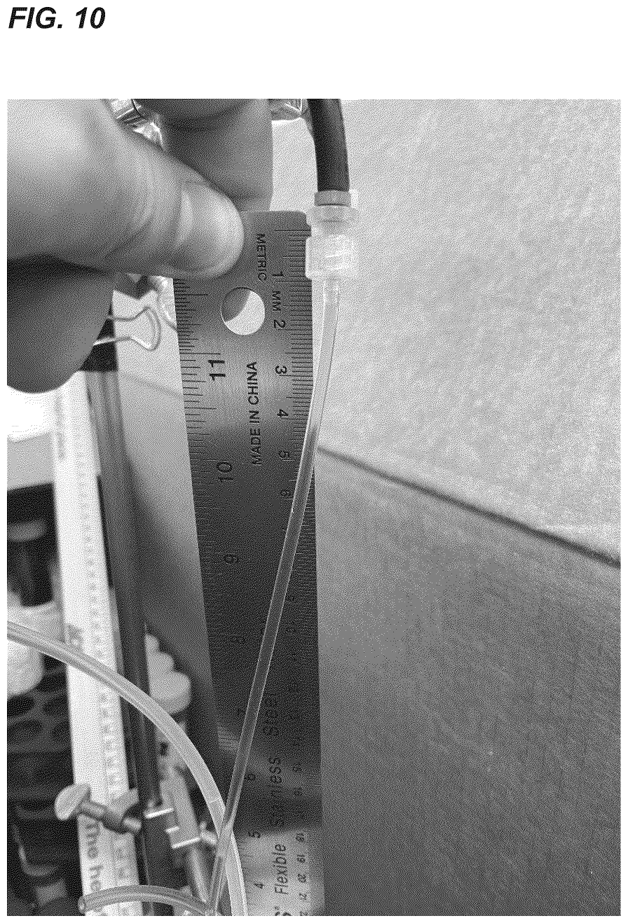

FIG. 10 illustrates an experiment demonstrating the diffusion of drug (orange colored) downward from a drug-releasing region into clear tubing.

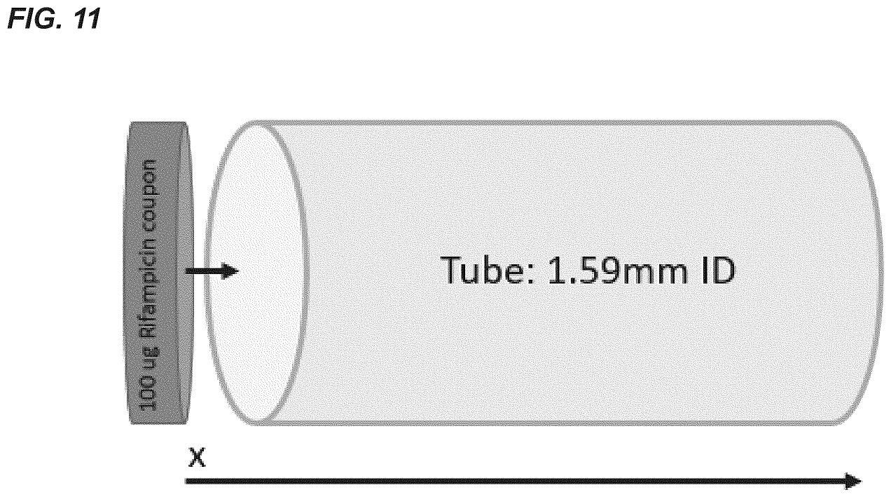

FIG. 11 schematically illustrates an experiment for diffusion into fluid contained in a horizontal tube, from a solid disc of drug and polymer.

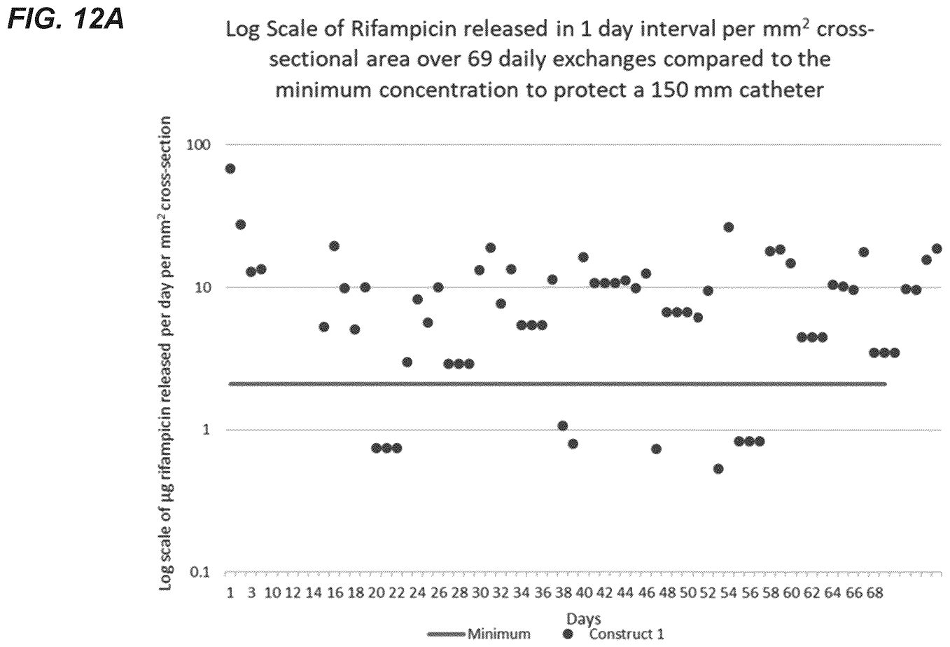

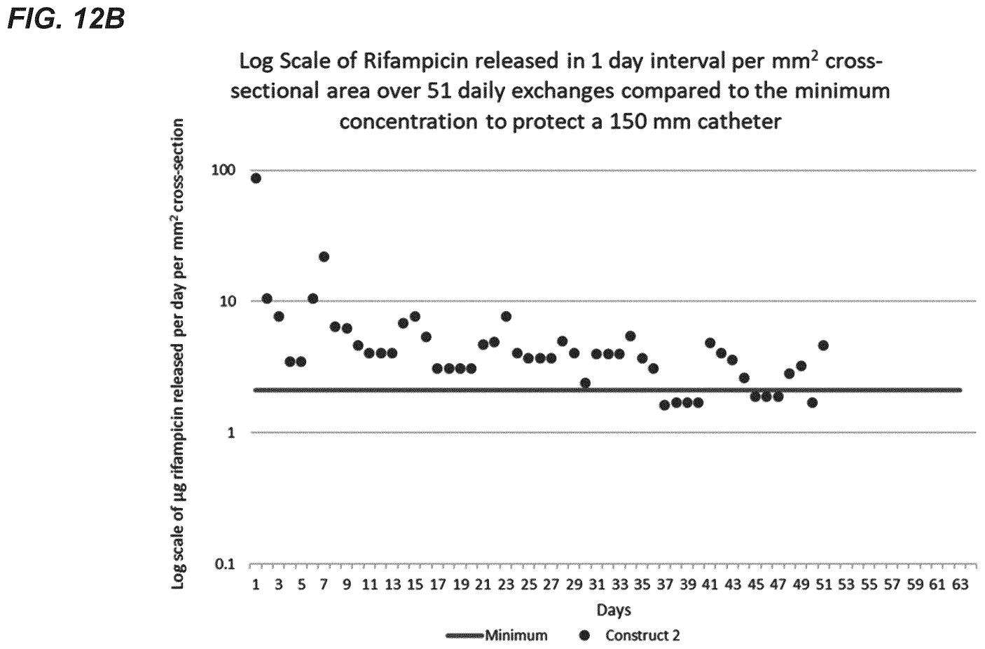

FIGS. 12A-B illustrate, for rifampicin, data similar to the data of FIGS. 9A and 9C, while comparing that data to the concentration that would be needed for diffusion to transfer enough drug to protect a catheter whose length is assumed to be 150 mm.

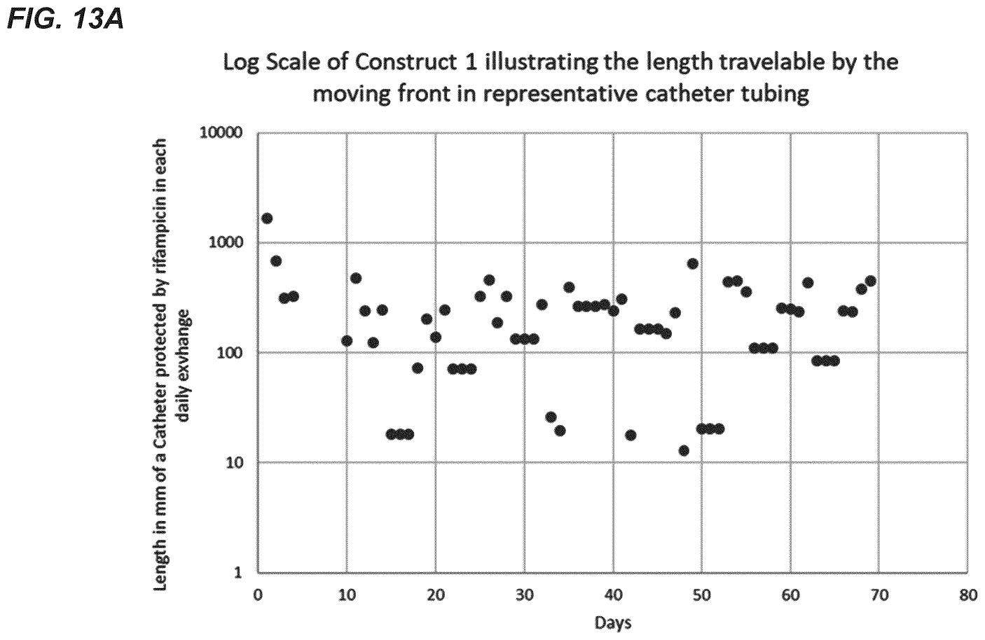

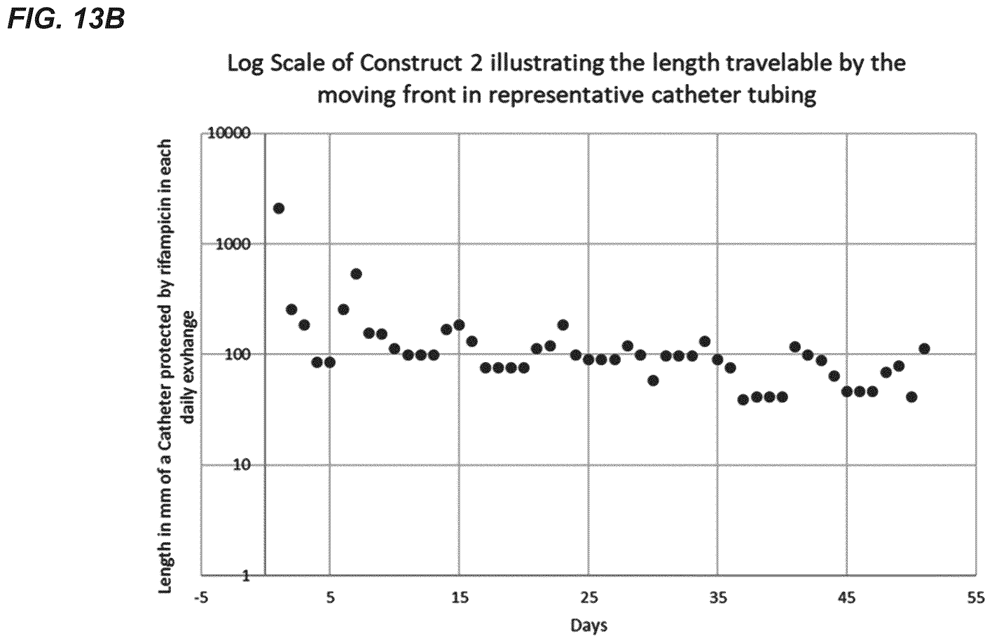

FIGS. 13A-B illustrate, for Constructs 1 and 2, the distance travelable by rifampicin in a catheter.

### DETAILED DESCRIPTION OF EMBODIMENTS OF THE INVENTION

Referring now to FIG. 1A, there is shown a prior art catheter assembly 10 comprising a catheter 12 and a catheter extension set 14, such as is currently used with almost all central venous catheters and with other types of catheters. At the time that such a catheter assembly is delivered to the user, such a catheter extension set is typically already permanently attached to a catheter that is suitable to enter a patient's body. What is illustrated in FIG. 1A shows a catheter extension set 14 having two branches 16 and 18. The catheter 12 and catheter extension set 14 can be used for a two-lumen catheter, with one tube being in communication with each lumen of the two-lumen catheter. In such a catheter, each extension tubing is attached to a separate lumen in the catheter.

As shown in FIG. 1A, such a catheter extension set is frequently used in combination with a cylindrical rod positioned outboard of a clamp, with the cylindrical rod releasing an antimicrobial substance. In the commercial product ClearGuard, the antimicrobial substance is chlorhexidine, which must be kept isolated from the patient's bloodstream through the use of a clamp 24 and appropriate procedures. For example, the cap 20 can include portions 22 coated with chlorhexidine, a broad-spectrum antimicrobial agent.

Also, as an example of known devices, referring now to FIG. 1B, there is shown a conventional catheter 30 made of a material that contains an antibiotic. The exemplary catheter 30 is a urinary catheter, which can be a type of catheter according to the present disclosure.

### Terminology

A catheter is a tubular device part of which is suitable to enter a patient's body and be in communication with bodily fluids. During use, a portion of the catheter typically exists inside the patient's body, while another portion exists outside the patient's body. A catheter can be single-lumen or multi-lumen.

Referring now to FIGS. 2A-2D, some embodiments of the invention can comprise a catheter assembly 2000 comprising a catheter 900 and a catheter extension set 1000. Some embodiments of the invention can comprise a catheter extender-lengthener 50 that is suitable to mate with a catheter or catheter extension set 1000. A catheter extender-lengthener 50 can have some commonalities with a catheter assembly 2000 comprising a catheter 900 and a catheter extension set 1000, but there also can be some differences.

A catheter extension set 1000 is considered to be an assembly of connectors and tubing that is permanently connected to the catheter 900 itself at the time the catheter assembly 2000 is delivered to the customer or hospital. If a catheter 900 is a single-lumen catheter, the catheter extension set 1000 generally has a single tube. If a catheter 900 is a multi-lumen catheter, the catheter extension set 1000 likely has a number of branches equal to the number of lumens in the catheter 900. At the end away from the catheter 900, each branch of the catheter extension set 1000 terminates in a respective hub that can be connected to or punctured by other medical devices.

A catheter extender-lengthener 50 is considered to be a separate item that can be optionally connected to a catheter 900 or to a catheter set 1000 when desired. A simple catheter extender-lengthener 50 can comprise a first connector 100 at a first end, and a tube 200 adjoining the first connector 100, and a second connector 300 adjoining the tube 200 at the end opposite the first connector 100. Such a simple embodiment of catheter extender-lengthener 50 can be used with a single-lumen catheter or catheter extension set, especially in circumstances when the catheter 900 is already implanted in a patient. A characteristic of a catheter extender-lengthener 50 may be that it is manufactured separately and can be sold separately from the catheter extension set 1000 or catheter 900 or catheter assembly 2000, and therefore its composition and design and other properties can be chosen separately from such choices for the catheter 900 or catheter extensions set 1000 or catheter assembly 2000. It also is possible that a catheter extender-lengthener can include branching or a “Y” configuration or limbs. A multi-limbed catheter extender-lengthener 60 may comprise a first connector 100, and a plurality of tubes 200 adjoining the first connector, and respective second connectors 300 adjoining each tube at the end opposite the first connector.

Another term sometimes used is a transfer set, especially in regard to hemodialysis and peritoneal dialysis. A transfer set can be considered to be similar to a catheter extender-lengthener 50, especially a single-lumen catheter extender-lengthener 50. However, a transfer set might be longer than a typical catheter extender-lengthener 50.

### Catheter Extender-Lengthener

Referring now to FIG. 2A, in an embodiment of the invention, there may be provided a catheter extender-lengthener 50 comprising a first connector 100 at a first end, and a tube 200 adjoining the first connector 100, and a second connector 300 adjoining the tube 200 at the end opposite the first connector 100. These components may define a continuous lumen extending through the first connector 100, the tube 200 and the second connector 300. What is illustrated in FIG. 2A is a single-lumen catheter extender-lengthener 50. It is also possible that exiting the first connector 100 there may be an additional tube 200, with each tube 200 having a respective second connector 300 at its end opposite the first connector 100, similarly defining a lumen therethrough.

In embodiments, the catheter extender-lengthener 50 may have geometric features suitable to interface with a known fitting at the end of a known catheter or with a catheter extension set 1000. A catheter used in combination with embodiments of the invention could in general be any catheter that is capable of carrying fluid flow, such as a catheter for hemodialysis, a catheter for peritoneal dialysis, a catheter for chemotherapy, a central venous catheter, a urinary catheter, or a catheter or tubing for any other purpose. Accordingly, the first connector 100 (at the first end of the tube 200) may be complementary with and mateable to the known fitting on a corresponding catheter 900 or catheter extension set 1000. The second connector 300 (at the second opposed end of the tube 200) may be identical to the known fitting on a corresponding catheter 900 or catheter assembly 2000 (or may, in all functional aspects, be equivalent to the known fitting on a corresponding catheter 900 or catheter assembly 2000). Similarly, the first connector 100 and the second connector 300 may have geometric properties such that the first connector 100 and the second connector 300 are able to connect with each other. As a result, the procedures for hospital personnel in interacting with the known fitting on the end of the known catheter 900 or catheter extension set 1000, and the procedures for hospital personnel in interacting with the second fitting 300 (attached to the second end of the catheter extender-lengthener 50) when the catheter extender-lengthener 50 is installed, may be identical. Such procedures may involve connecting a cap, connecting an intravenous infusion line, connecting a dialysis line, or connecting any other medical component. Appropriate connectors or adaptors may be provided for use with such other procedures or equipment, if needed.

In an embodiment of the invention, the first connector 100 and the second connector 300 may have geometric properties such that the first connector 100 and the second connector 300 are in some manner opposite of each other or complementary to each other. For example, one of the connectors 100, 300 could be a male connector and the other could be a female connector. This means that the associated catheter fitting that a nurse would have to connect to another medical device in the absence of catheter extender-lengthener 50 may be the same as the fitting that a nurse would have to connect to another medical device in the presence of catheter extender-lengthener 50. Alternatively, the fittings or connectors on the respective ends of the catheter extender-lengthener 50 could be any other desired combination of fittings.

In embodiments, fittings on the ends of the catheter extender-lengthener 50 can be Luer lock fittings, which are commonly used in health care settings, or can be any other desired type of threaded fitting or other type of fitting.

In an embodiment of the invention, catheter extender-lengthener 50 could have just one branch or could have more than one branch, each branch having its own tube 200 and ending in a respective second connector 300. In such a situation, the respective tubes 200 could be identical to each other or could differ from each other in any way desired including geometry, composition, drug content, drug concentration, or any other feature.

In catheter extender-lengthener 50, tube 200 could be provided with a clamp 2600 that is movable between an open configuration and a closed configuration. The clamp 2600 could be geometrically trapped on tube 200 so that clamp 2600 is not able to be removed from tube 200. Alternatively, if desired, the clamp 2600 could be removable from the tube 200. It is possible that clamp 2600 could be used to clamp at any of various positions along the length of tube 200, or, alternatively, there could be design features such that it is only possible for clamp 2600 to clamp on tube 200 at one or a limited number of discrete locations. Clamp 2600 may operate by squeezing one portion of the circumference of tube 200 against an opposite portion of the circumference of tube 200 to form a generally flat compressed tube region that does not permit the passage of fluid therethrough from one side of the clamp 2600 to the other side of clamp 2600. Known designs of clamps include ratcheting pinch type clamps as illustrated in FIG. 2A; roller clamps; and slotted clamps having a slot of varying width as illustrated in FIG. 2B.

### Drug Release Characteristics

In an embodiment of the invention, the tube 200 may be made of or may comprise a drug-eluting polymer. It can be noted that the connectors 100, 300 may be relatively rigid and the tube 200 may be relatively flexible. It is disclosed in U.S. Pat. No. 7,347,853, in which the hub is referred to as an “extension leg unit,” that the “extension leg unit,” which is rigid, may contain drug. In embodiments of the present invention, the tube 200 itself may be drug-eluting. It is also possible that the first connector 100, which may also be called a hub, or second connector 300, could also contain a drug, but it is not necessary for the hub or first connector 100 to be drug-eluting or for the or second connector 300 to be drug-eluting. The materials requirements, stiffness, etc. may be different for the tube 200 as compared to an “extension leg unit,” with for example the “extension leg unit” typically being much stiffer or mechanically stronger than a tube 200. Also, the tube 200 may provide greater internal surface area for drug release from the relatively rigid components.

In an embodiment of the invention, the catheter extension tube 200 may comprise a polymer material that comprises or is mixed with an antibiotic such as minocycline and rifampin. The use of two drugs, such as minocycline and rifampin in combination, is accepted medical practice. The two drugs can be used in combination with a polymer material that is known to be acceptable in regard to biocompatibility. In addition to minocycline and rifampin, generally any antibacterial drug or drug combination is contemplated. The material of which the tube 200 is made could comprise silicone or polyurethane or EVA (ethylene vinyl acetate), each of which is available in various grades, or other elastomeric or polymeric material as known in the art, and an antibiotic. EVA or other material could be combined with release-modifying agents such as polyethylene glycol or polycaprolactone or both. As described elsewhere herein, the combination of the two drugs and the polymer(s) may be suitable to be molded or extruded to form desired shapes including tubing. In an embodiment of the invention, the polymer and the antibiotic(s) and release-modifying agents (if used) may be blended with each other during melt-processing, and then that material may be formed into tubing, as described in the parent patent application. In yet another embodiment of the invention, the antibiotic(s) or other substance may be deposited or incorporated in the polymer by solvent deposition (infusion), which is a technique in which the substance is dissolved in a solvent to form a solution, and the solution is then infused into the polymer, and the solvent is then allowed to evaporate. Solvent deposition (infusion) may be appropriate for use with drugs that are temperature-sensitive and might decompose or be damaged by the temperatures encountered during melt-processing. In embodiments of the invention, coatings may be used. In embodiments of the invention, the question of biocompatibility of the extension tubing material can be even easier because the extension tube may be used outside the patient's body.

In an embodiment of the invention, it is possible for the concentration of minocycline in the material to be as large as 30% (w/w), or 20% (w/w), or 10%, or 5%, or 2% or any values in between. It is possible for the concentration of rifampin in the material to be as large as 30% (w/w), or 20% (w/w), or 10%, or 5%, or 2% or any values in between. It is possible for each of these concentrations to be as large as 30% (w/w), or 20% (w/w), or 10%, or 5%, or 2% or any values in between. In contrast, the material disclosed by Darouiche et al. (New England Journal of Medicine, vol. 340, no. 1, pp. 1-8, Jan. 7, 1999) contains minocycline and rifampin impregnated into catheter at a concentration of only 1.4% (w/v). This more limited concentration limits the amount of time that such a catheter can usefully elute drug.

A therapeutic concentration of a drug in a fluid can be considered to be a defined multiple of the Minimum Inhibitory Concentration for growth of a microorganism of interest, or the Minimum Inhibitory Concentration for biofilm. Such multiple can, for example, be 10 or 20 or more, as desired, for inhibition and for preventing the development of resistant strains.

In embodiments of the invention, it is possible that the polymer and the drug(s) and possibly other substances can co-exist in a material by mixing at a molecular level, or in the form of particulate blended with surrounding material, or in the form of a co-continuous network, or in discrete phases, or mixed among each other in any other configuration.

It is possible to make an entire catheter 900 out of special material such as drug-eluting polymer. However, with use of an embodiment of the invention, this is not essential. Instead, it is sufficient to provide the special material mostly near or at the site of insertion through the skin or outside the insertion site such as at the catheter extension set 1000 or in the catheter extender-lengthener 50. Thus, it is possible to just protect those locations, and so it is possible to only make the tube 200 out of drug-eluting polymer. It would be more expensive and difficult to make the entire catheter 900 out of the drug-eluting polymer. Also, if the catheter 900 and the catheter extension set 1000 are made separately and then are permanently attached to each other, the material choice for the catheter 900, or that portion of the catheter 900 that enters the patient's body, can be a separate decision from the material choice for the catheter extension set 1000.

Similarly, in the situation of a catheter extender-lengthener 50 that comprises the special material, it is not necessary to use the special material for either the catheter 900 itself or the catheter extension set 1000. This allows the material for each of these entities to be chosen for optimum mechanical or other properties for its individual intended purpose. Also, as discussed elsewhere herein, it is furthermore possible within those entities to make different portions of the tubing of different materials.

With the drug-eluting catheter extender-lengthener 50 of embodiments of the invention, no change of practice or protocol would be needed to compared use of an ordinary catheter extender-lengthener 50 or other catheter-related components. FIG. 2A shows a catheter extender-lengthener 50 of an embodiment of the invention. In an embodiment of the invention, the tubing 200 could be made of or could comprise the described drug-eluting polymer.

In embodiments of the invention, it is possible to provide antibacterial release from the catheter extender-lengthener 50 for a duration such as 69 days. The material of which the tube 200 is made could comprise silicone or polyurethane or EVA (ethylene vinyl acetate) (each of which has various possible grades or formulations) or polytetrafluoroethylene or latex rubber or other rubber or other material as known in the art, and an antibiotic. EVA could be in combination with release-modifying agents such as polyethylene glycol or polycaprolactone. Polyurethane catheter materials, which are in current use, are known to be robust in terms of mechanical properties.

In current dialysis practice, for certain situations such as acute dialysis there is used a catheter that might not have a cuff and is designed to be used only for approximately one week. When chronic catheters are being placed for dialysis, often they are placed because the patient does not yet have a workable fistula. In this situation, in most instances the plan of the physician is that the catheter would be removed in a month or so when the access fistula is evolved and ready for use. However, it can also be noted that there are at least 5%-10% of dialysis patients for whom fistulas and grafts never work. Thus, there remains a need for catheter technology for longer-duration placement of catheters for hemodialysis. There is also a similar need regarding peritoneal dialysis.

An antibiotic incorporated into the tubing can diffuse into liquid in the lumen of the tube and can diffuse in both directions from the clamp 2600. This will provide antibiotic exposure to the entire lumen of the catheter 900, from hub to tip. In the use of catheters, a cuff (either a superficial cuff or a cuff positioned in subcutaneous tissue) may provide some anchoring of a percutaneous catheter, to prevent motion along the length direction of the catheter. A cuff may, for example, be made of Dacron felt material allowing tissue ingrowth creates fibrous block. The use of embodiments of the invention may help to maintain sterility near the cuff and also where the catheter passes through the skin, so that a catheter could last for months or even longer without infection at the exit site or the lumen.

Catheter Extender-Lengthener Used with Clamp in any Location, not Requiring all the Antibiotic to be in an Isolated Region

In embodiments of the invention which use antibiotics such as minocycline and rifampin, it is acceptable if some of the antibiotic reaches the patient's blood stream, because the general use of antibiotics ordinarily does involve release into the patient's body. If embodiments of the invention contain minocycline and rifampin and if those antibiotics come into contact with blood such as when tubing is squeezed, that is acceptable. For example, a nurse could inject sterile saline into the patient through the catheter extender-lengthener 50, with the saline absorbing some antibiotic during its passage through the tube 200. Similarly, if a closure cap 2800 with a projection 2850, resembling ClearGuard, was (as in embodiments of the invention) impregnated with antibiotics (rather than antiseptic), it would be acceptable for a nurse to squeeze the tube 200, and it would be acceptable if such antibiotic material travels further into the catheter extender-lengthener 50 or catheter extension set 1000 or catheter 900. That would be acceptable because such antibiotic does not cause harm if it enters the patient's body. The same is true for an assembly of a catheter 900 and a catheter extension set 1000, in which the catheter extension tubing (or a portion thereof) in the catheter extension set 1000 is drug-eluting.

In an embodiment, it is possible for the tubing to have a clamp 2600 disposed thereon, wherein the tubing comprises markings or indicators indicating locations of the clamp disposed on the tubing that correspond to respective situations or quantities of release of the antimicrobial substance from the projection 2850 of cap 2800 to fluid on one side of the clamp 2600 or an opposed side of the clamp 2600.

In an embodiment of the invention, the clamp 2600 may be a pinch clamp, as shown in FIG. 2A, which pinches the tube 200 shut by pressing one portion of the circumference of the tube 200 against an opposite part of the circumference of the tube 200.

In an embodiment of the invention, the clamp 2600 may be confined to a certain position or range of positions along the tube 200, such as by the connectors, by dimensional relationships or by external features on the tube 200 or by other geometric features. Such limit or confinement may help to define which volumes of liquid are exposed to elution of substances from the catheter extension tube 200. In FIG. 3A, it is shown that a clampable region is located centrally in the catheter extender-lengthener 50, and on each side of the clampable region there is a drug-releasing region. In such a situation, drug could be released to the fluid region on one side of the clamp 2600, which may be isolated by the clamp 2600, and drug also could be released to the fluid region on the other side of the clamp 2600, which may be in fluid communication with the patient's bodily fluids. In FIGS. 3B and 3C, there is a clampable region but there is a drug-releasing region only on one side of the clamp 2600, rather than on both sides of the clamp 2600. The drug-releasing region could be either on the side away from the patient, which would keep the released substance out of contact with the patient, or it could be on the patient side, which would allow the released substance to be in fluid communication with the patient's bodily fluids. In general, the clampable region itself could be either drug-eluting or non-drug-eluting.

### Co-Extrusion or Multi-Layer Wall of Catheter or Tube

As described elsewhere herein, it is possible to make the entire catheter extension tube 200 out of a single material. Alternatively, it is possible that some portions of the catheter extension tube wall could be compositionally different from other portions.

In regard to drug release characteristics, as described elsewhere herein, a favorable host polymer is EVA (ethylene vinyl acetate) copolymer (Elvax, available from Dow Chemical, formerly DuPont) with release-modifying agent material including polyethylene glycol (PEG) and possibly also polycaprolactone (PCL). It is believed that this material has appropriate mechanical properties such that it could be considered as a material for use in making a catheter 900 or catheter extension set 1000 or catheter extension tube. The working environment of catheters 900 and tubing such as the tube 200 would include handling, fluid exposure and also repeated clamping and release of clamping. There is already a substantial base of experience in making catheters and similar products from polymers such as silicone and polyurethane and elastomers in general.

One possible embodiment is to make inventive components such as the tube 200 from silicone or polyurethane as a major material (rather than ethylene vinyl acetate as disclosed elsewhere herein), with the addition of the desired drugs and possibly release modifying agents.

In embodiments of the invention, the tubing material properties and dimensions may be chosen to meet desired mechanical requirements. For example, the tubing may have a tensile strength of at least 3 lbf, which is typical for such uses, or may have a tensile strength of at least 8 lbf, which is typical of catheters. The tubing material also may be chosen so that the tubing can be clamped flat by a pinch type clamp a desired number of times without suffering any mechanical damage after a desired number of clamping cycles. The tubing material also may be chosen so that when the tubing is clamped flat by a pinch type clamp, the inner wall of the tubing does not stick to the opposed portion of the inner wall, and the tubing springs back sufficiently when the clamp is released.

Referring now to FIG. 4A, in yet another possible embodiment, it is possible that the tube 200 can be made by co-extrusion or another appropriate process such that it comprises an inner layer of one material 71 and an outer layer of a second different material 72. The two layers can have an annular concentric geometry and can surround a lumen. The inner layer 71 (which would be exposed to the fluid in the lumen) can be the described EVA (ethylene vinyl acetate) family of materials containing antibiotic or similar material as well as release modifying agent(s). In such a situation, the inner layer 71 may be made of or comprise the material of embodiment of the invention which is able to release minocycline and rifampin or generally any antibiotic, antibacterial or other desired substance. Outside of and surrounding this layer 71 can be provided an outer layer 72 of a more typical catheter material, perhaps not containing antibiotic. In such an article, the antibiotic in the catheter extension set 1000 or catheter extension tube would not be exposed to outside handling, to health care workers etc. The outer layer 72 may be made of or comprise a material that is familiar for use in catheters such as silicone, polyurethane, or other known materials that have a base of experience with catheter manufacturing, handling, clamping etc. It is not thought that there would be any problem with having EVA on the outside surface of the tube or as the only base material of which a catheter or tube 200 is made, but in an embodiment it would be possible to use an outer layer of silicone, polyurethane or other such materials because there already is a large base of experience with making and using catheters, catheter extension sets etc. made of such materials.

It can be noted that any of various polymer processing methods could be used, such as co-extrusion, injection molding, three-dimensional printing, polymer welding or other known processes.

In yet another embodiment, referring now to FIG. 4B, it is further possible that a catheter extender-lengthener 50 could have a tube 200 such that a portion 200′ of the length of the tube 200 contains, as described herein, an antibiotic-releasing polymer in any geometry (which could be the just-described annular layered geometry or could be entirely made of the antibiotic-polymer combination), and another portion 200″ of the length of the tube 200 does not contain antibiotic-releasing polymer. The two portions 200′ and 200″ of the tube 200 could be joined to each other by polymer welding, adhesive, or any other known joining process. The catheter extender-lengthener tube 50 may comprise a third portion of its length that comprises another material that comprises antibiotic.

It is possible to provide any geometric arrangement of segments and any number of segments along the length of the tube, with different segments having different compositions. Any of the described variations in composition or design of the tube could be used with any of the devices described herein.

### Catheter Assembly Comprising a Catheter and a Catheter Extension Set

Referring now to FIG. 5, in an embodiment of the invention, there may be provided a catheter assembly 2000 comprising a catheter 900 and a catheter extension set 1000, which may be permanently joined to each other.

Catheter 900 may be suitable to be placed inside or partially inside a patient's body. There may also be provided a catheter extension set 1000. At one end, catheter 900 may be joined to catheter extension set 1000 by a junction 950. Junction 950 typically may be a permanent connection between catheter 900 and catheter extension set 1000. Such junction may typically be made before the catheter assembly 2000 is delivered to a user.

Catheter 900 may be a single-lumen catheter or a multi-lumen catheter. For a catheter 900 that is a single-lumen catheter, typically the extension set 1000 may have only one tube 200, which may end in an end connector. There may be a lumen through the junction 950, the tube 200 and the end connector, with the lumen being in communication with the lumen in the catheter 900. For a multi-lumen catheter 900, there may be a tube 200 corresponding to each lumen of the catheter 900, with each tube having a respective end connector. In a multi-lumen catheter assembly 2000, for each tube, there may be a lumen through the junction 950, the tube 200 and the end connector, with each lumen being in communication with the respective lumen in the catheter 900.

Each end connector may be suitable to engage with a respective cap 2800 suitably to close off passage of fluid therethrough. Cap 2800 may be a simple inert cap or may be a cap that is suitable to elute a desired substance, as discussed elsewhere herein.

Tubes 200 may be provided with respective clamps 2600 having an open position and a closed position, such that when clamp 2600 is in the open position there is fluid communication through the lumen from one side of the clamp 2600 to the other side of clamp 2600, and when clamp 2600 is in the closed position there is no fluid communication across the clamp 2600. Clamp 2600 may be trapped between connectors or junctions at the ends of tube 200, so that clamp 2600 cannot slide off of the tube 200. It may be possible for clamp 2600 to occupy a variety of positions along the length of tube 200. Alternatively, the position of clamp 2600 along tube 200 may be geometrically constrained.

In an embodiment of the invention, as described elsewhere herein, it is possible that tube 200 may elute a desired substance on one side or on both sides of clamp 2600, even when clamp 2600 is in its closed position. Eluted substance on one side of clamp 2600 may enter an isolated volume of fluid, while eluted substance on the other side of clamp 2600 may be in communication with bodily fluids. In an embodiment of the invention, the position of clamp 2600 along the length of tube 200 may influence how much surface area of the lumen of tube 200 is exposed to the isolated fluid and how much surface area of the lumen of tube 200 is exposed to bodily fluids. So, if the clamp 2600 is located such that most of the length of the tube 200 is between the patient and the clamp 2600, then more of the eluted substance will be exposed directly to the patient. If the clamp 2600 is located such that most of the length of the tube 200 is between the clamp 2600 and the cap, then more of the eluted substance will be directed to the isolated volume of fluid. This fact can be used to adjust the drug delivery characteristics as desired. The tube 200 may comprise gradations or markings to indicate placement of the clamp for various purposes or various drug delivery characteristics.

It is found that the polymer EVA in combination with various additives as described herein is suitable for the manufacture of tubing, but the material is somewhat soft and the springback that it exhibits after being pressed flat during clamping is less than the springback of certain other possible tubing materials. Depending on the design of the clamp 2600, it is possible that the clamp 2600 can be geometrically constrained to remain located in a certain location or range of locations on tube 200. Accordingly, yet another possibility is that, if different segments of the tube 200 are made of different materials, the portion of the tube that is intended to be clamped can be made of a conventional material that is suitable or optimal for being clamped, and a portion of the tube that is intended not to be clamped can be made at least partially of the drug-releasing material described herein.

Embodiment in which Antibiotic-Containing Material is Present in a Projection

In still other embodiments of the invention, it would be possible to make the described material into a shape of a projection 2850 such as a rod that is connected to an external cap 2800, that is suitable to occupy a portion of the interior volume of the catheter extension set 1000 or the interior volume of the catheter extender-lengthener 50. Such a projection 2850 could be geometrically similar to the rod in the ClearGuard product, but containing antibiotic instead of chlorhexidine. In an embodiment of the invention, the polymer and one or more antibiotics could be placed in the projection 2850. Such projection 2850 could be generally round (cylindrical) in cross-section, as is typically done, but it is also possible that the projection 2850 could have other cross-sectional shapes. For example, in embodiments of the invention, it would be possible to make a projection 2850 that is soft and compressible similar to the catheter 900 itself and suitable to be squeezed by the walls of the tubing as the walls of the tubing are squeezed by a clamp 2600. Such an embodiment could be used in combination with a catheter extender-lengthener 50 or with a catheter extension set 1000. It is furthermore possible that the projection 2850 could contain both antibiotic and some other substance such as an antimicrobial.

As described, it is possible that the projection 2850 could be inserted into the interior of a catheter extension set 1000 or a catheter extender-lengthener 50, as illustrated in FIG. 6. In some instances, the projection 2850 could end before (outboard of) the clamp 2600, and in other instances the projection 2850 could proceed through the clamped region.

Referring now to FIGS. 6 and 7, there is shown a projection 2850 that is generally cylindrical and occupies space inside the tubing 200, and there is also shown a clamp 2600. However, the clamp 2600 that is shown in FIG. 7 is not a pinch clamp as shown elsewhere herein, but rather is a clamp that applies force generally in a radial direction around generally the entire circumference of the tube 200. In this design, it is possible for the projection 2850 to extend through and beyond the clamped region and to exist in the portion of the tubing that is in fluid communication with bodily fluids. This would be allowable if the substance is an antibiotic or generally any substance that is non-toxic to living tissue. Other shapes for projection 2850 are also possible, including projections 2850 having a cross-sectional shape that is flattened rather than round. Various geometries of clamp 2600 are also possible. If the projection is made in flattened shape with tapered edges (like a knife edge), and if it is made of a malleable or elastic material, it is possible that a standard click clamp might work to clamp and occlude the extension tubing whether the projection is in place or not. FIG. 6 also shows a “butterfly” shaped handle 80 that can be attached to the catheter extender-lengthener 50 and can be used for gripping or manipulating the catheter extender-lengthener 50 or for anchoring it such as by taping it to a patient's skin. FIG. 7 shows a clamp suitable to seal around a round rod-shaped projection 2850. However, it can be noted that when the projection 2850 is absent from the tubing, such a clamp would not be suitable to close the tubing and so a second different clamp would be needed for many catheter functions.

The tube 200 may be suitable to be compressed by a clamp 2600 suitably so that when the clamp 2600 is closed, passage of fluid through the tube 200 from one side of the clamped region to the other side of the clamped region is substantially blocked. The tube 200 may be suitable to be clamped somewhere along its length. In such a situation, some liquid may be trapped away from the patient in space between the clamp and the closure cap of the tube 200. This liquid may receive drug or beneficial substance eluted from the tube 200. In such a situation, some other liquid may exist inside the tube 200 or inside the catheter extension set 1000 or the catheter 900, on the side of the clamp 2600 that is toward the patient. This liquid also may receive drug or beneficial substance eluted from the catheter extension tube 200. Mass transfer within such liquid is discussed elsewhere herein.

In the described tube 200, even when the tube 200 is clamped (which occurs a large portion of the time), the internal surface of tube 200 that is on the patient side of the clamp is likely still in contact with bodily fluids and may be releasing antibiotic or other beneficial substance approximately continuously, and that released substance may be able to travel toward the patient all during that time even when the clamp is closed, and thereby protect portions of the catheter extension set 1000 or catheter 900 that are on the patient side of the clamp 2600.

In embodiments, it is possible to create the tube 200 which has a configuration suitable so that it can be clamped in a way that leaves antibiotic-containing polymer on the patient side of the clamp 2600, exposed to the interior of the catheter extension set 1000 and able to release drug antibiotic etc. into the fluid that is present in that region of the catheter extension set 1000. Of course, it is possible that antibiotic-containing polymer could also be exposed to whatever fluid is present inside the catheter extension set 1000 on the nurse side (back side) of the clamp 2600, which is isolated fluid.

The tube 200 may be such that when the clamp 2600 is removed, the tube 200 reopens by virtue of its elasticity and it is possible for fluid to pass through the tube 200 from one side of the now-unclamped region to the other side of the now-unclamped region.

In embodiments, the projection 2850 could contain more than one category of substance, e.g., both antibiotic and antimicrobial. Appropriate procedures would have to be followed.

It should be understood that all of the lengths discussed herein are arbitrary. For example, it would also be possible to add a catheter extension set 1000 and not modify the length of a conventional part of a catheter 900 or catheter extension set 1000. The length of the catheter extender-lengthener 50 can be any length desired.

### Use of Combinations of Devices

In still other embodiments of the invention, and referring now to Table 1, it would be possible to use the described drug-releasing material for the wall of the tube 200, as already described, further in combination with an antibacterial catheter lock such as citrate with benzyl alcohol, or in combination with a cap 2800 that contains an antibacterial-eluting material. Similarly, it is possible to use the described embodiments of the invention for a catheter extender-lengthener 50, and later, such as when the drug elution from the tube 200 tapers off, it would be possible to continue to use the same catheter extender-lengthener 50 for a somewhat longer time by using a cap 2800 having an eluting projection 2850 to provide longer protection. As yet another alternative, it would be possible, during use, to replace the catheter extender-lengthener 50 with another catheter extender-lengthener 50. The replacement catheter extender-lengthener 50 could comprise the same drug or drug combination as the earlier catheter extender-lengthener or could comprise a different drug or drug combination, or a different concentration or dose of drug. The use of a drug-eluting catheter extender-lengthener 50 provides an opportunity to easily change the composition or concentration or dose of a drug that is being administered to help maintain sterility of the catheter extension set 1000 or catheter 900. As yet another alternative, it would be possible to use an antibiotic-eluting cap 2800 either instead of a catheter extender-lengthener 50 or in combination with a catheter extender-lengthener 50. Furthermore, the cap 2800 could elute either an antibiotic or an antimicrobial or both, as long as appropriate protocols are followed.

TABLE 1
Strategies for Addressing Catheter Related Blood Stream Infections
(CRBSI)

| Extent of | Presence | Presence of | Presence of | Presence of |
|---|---|---|---|---|
| aggressiveness | of | antibiotic in | antibiotic in | antibiotic |
| in combatting | antibiotic in | Catheter | Extender- | in |
| CRBSI | Catheter | extension set | lengthener | Cap |
| Most | Yes | Yes | Yes | Yes |
| aggressive | | | | |
| More | | Yes | Yes | Yes |
| aggressive | | | | |
| Intermediate | | | Yes | Yes |
| Least | | | | Yes |
| aggressive | | | | |

An embodiments of the invention may comprise: choosing antibiotics or antiseptics that do not have systemic toxicities; creating a material (polymer) that contains a significant reservoir of the antibacterial or antibiotic material; using that material as the catheter extension set 1000, while using that same material to make the entire catheter 900 or not using the same material to make the entire catheter.

It can be noted that in a conventional device that uses an antimicrobial substance (such as chlorhexidine) in a geometry such as ClearGuard, it is necessary to clamp the tube so that substantially all of the antimicrobial substance is kept out of contact with the patient's bodily fluids. In embodiments of the invention, which release antibiotic, it is not a problem if some of the antibiotic comes into contact with the patient's bodily fluids or enters the patient's body. Therefore, with embodiments of the invention, the position of the clamp 2600 within the length of the tube 200 can be more arbitrary, and it may be possible to use less strict protocols.

In an embodiment of the invention, it is possible to use various strategies to limit infection in a patient using embodiments of the described catheter extender-lengthener 50. As described herein, it is possible to implant in a patient a catheter 900, the catheter 900 being part of a catheter assembly 2000 that comprises the catheter 900 and a catheter extension set 1000. Either the catheter 900 or the catheter extension set 1000 or both may comprise tubing whose walls contain an antibiotic. It is then possible to monitor bodily indicators of the patient for evidence of Catheter Related Blood Stream Infection or other problems. Such monitoring can include monitoring bodily temperature, monitoring white blood cell count, or other forms of monitoring. If such bodily indicators indicate any problem, it is possible to then attach an antibiotic-containing catheter extender-lengthener 50 to an end of the catheter assembly 2000. If that is not effective, it would be possible to attach a different catheter extender-lengthener 50 containing a different antibiotic or concentration of antibiotic. Similarly, if elution from a given catheter extender-lengthener 50 declines, it would be possible to replace the catheter extender-lengthener 50 with a new catheter extender-lengthener 50. All of this could be influenced by monitoring bodily indicators of the patient.

### Materials and Compositions

As is also discussed elsewhere herein and in the parent patent application, in regard to drug release characteristics, a favorable host polymer is EVA (ethylene vinyl acetate) copolymer (Elvax, available from Dow, formerly DuPont) in combination with a release-modifying agent such as polyethylene glycol (PEG) and possibly also polycaprolactone (PCL). Suitable antibiotics include rifampin and minocycline and combinations thereof. It is believed that the mechanical properties of such material combination are suitable for use in making tubing. Other possible materials include silicone, polyurethane and variations thereof, polytetrafluoroethylene, latex rubber, other rubber, in combination with one or more antibiotics and possibly in combination with one or more release modifying agents.

In embodiments of the invention, in addition to the already-described drugs, other possible drugs that could be used include: minocycline; other members of the Tetracyclines class of antibiotics; rifampicin; Other antimycobacterial drugs; Ciprofloxacin; other fluoroquinolones; Vancomycin; other glycopeptides; Cephalosporins (for more gram negative applications); Penicillins; Gentamicin; other aminoglycosides; Sulfonamides; Macrolides; Carbapenems; linezolid; other oxazolidinones; daptomycin; other lipopeptides; and Immunosuppressants.

In an embodiment, the combination of the drug or drugs and the polymer material may be such as to provide desired release characteristics of the drug or drugs. The drug or drugs may be released to liquid that is in contact with the material.

In an embodiment of the invention, the combination of antibiotic(s) and catheter extension tube material may be such as to provide elution of the antibiotic or antibacterial substance(s) over a period of several weeks. Representative materials and antibiotics are further described elsewhere herein, and in U.S. provisional patent application Ser. No. 63/278,595 filed Nov. 12, 2021, and in U.S. nonprovisional patent application Ser. No. 17/986,604 filed Nov. 14, 2022, which are incorporated by reference herein. In the catheter extender-lengthener 50, the tube 200 can proceed from a first connector 100 or hub, and the first connector 100 or hub can be suitable to connect to a catheter extension set 1000 which is part of a catheter assembly 2000 comprising a catheter 900 that extends inside the patient's body. The use of the drug in tube 200 means that the drug-containing material does not need to be present in the form of the catheter 900 that actually extends inside the patient's body. The catheter extension set 1000 is typically located outside the patient's body and the catheter extender-lengthener 50 (if used) is typically located outside the patient's body. The tube 200 may contain antibiotic in addition to polymer and may elute antibiotics into the fluid that contacts it. The antibiotic could diffuse out of the material of tube 200 and migrate into the liquid inside the lumen of catheter extender-lengthener 50 or into the liquid inside catheter extension set 1000 and possibly into additional nearby places.

In embodiments of the invention, it is possible to achieve significantly long clinically useful duration of drug release, such as even up to 1.5 months. This would be a significant improvement in regard to catheter infections in dialysis patients and other patients. In embodiments of the invention, antibiotic may be present in the wall (bulk material) of the catheter extension tube 200. This makes available a larger quantity of antibiotic compared to depositing a coating on the interior surface of a catheter extension tube or even compared to impregnating a catheter extension tube with antibiotic dissolved in a solvent after manufacture of the catheter extension tube.

In some known catheters 900 and catheter extension sets 1000 and catheter assemblies 2000, the process of depositing drug in such items only deposits enough drug for elution for a duration of 2 or 3 days, with that duration being limited by the amount of drug that can be deposited into the material. In such situations, antibiotic is dissolved in a solvent and then is infused or deposited from solution into the polymer of the already-manufactured tubing and the solvent is allowed to evaporate. This is different from embodiments of the invention, because in embodiments of the invention the drug may be thoroughly mixed with polymer material early in the manufacturing process. The inventive process can deposit larger amounts of drug into the polymer than infusion or deposition using a solvent. Darouiche (New England Journal of Medicine, vol. 340 no. 1, Jan. 7, 1999, pp. 1-8) reports that the concentration of minocycline and rifampin achievable by this method is about 1.4% w/v. Deposition of desired substances in the form of a coating (rather than solvent deposition or infusion) also has limitations regarding how much material can be deposited and what duration of release can be achieved. Darouiche's publication reports that an earlier publication by Darouiche found a concentration achievable by means of a coating to be smaller, by a factor of several times, than what is achievable by solvent deposition (infusion). In contrast, embodiments of the present invention can release therapeutic amounts of drug for a duration in the range of approximately two months, because of the larger concentration of drug contained in the inventive material or device.

In an embodiment of the invention, the tubing may contain material such that the least one antibiotic is present in the material at a concentration of at least 2% by weight, or at least 3%, or 4%, or 5%, or 6% or 7% or 8% or 9% or 10% or 12% or 14% or 16% or 18% or 20% or 25% or 30%. This can be true for minocycline or for rifampin or for both. In an embodiment of the invention, the polymer and the at least one antibiotic may be blended together in the material. Such blending may occur at an elevated temperature suitable to melt or at least soften the various ingredients. In an embodiment of the invention, the at least one antibiotic may deposited in the polymer by solvent deposition (infusion). Solvent deposition (infusion) may be appropriate for antibiotics that would be damaged by elevated temperatures associated with polymer processes such as hot melt extrusion. In an embodiment of the invention, the catheter extender-lengthener 50 or the catheter extension set 1000 or the catheter assembly 2000 may be capable of releasing a therapeutic amount of the at least one antibiotic, to a fluid contained within the tube, for at least 30 days.

Mass Transfer Processes and Drug Delivery to Protect Entire Catheter from Becoming Infected

It is worthwhile to discuss the mass transfer processes that can take place during use of embodiments of the invention. If the catheter extension set 1000 or the catheter extender-lengthener 50 contains drug-releasing polymer on the patient side of the clamp 2600, drug can be expected to be released into whatever fluid is contained inside the tube on the patient side of the clamp 2600, which is isolated fluid. Because of the clamp 2600, such fluid will be generally or macroscopically stagnant. Drug release into the isolated fluid will, first of all, have an effect of microorganism killing and biofilm discouragement within the isolated fluid region itself.

However, such effects within the isolated fluid region are not the only expected benefit. It is also possible that fluid on the patient side of the clamp 2600 may receive drug from drug-containing tubing wall. It is appropriate to also consider what mechanisms of mass transfer exist to move the antibiotic or other desirable substance beyond the catheter extender-lengthener 50 or the catheter extension set 1000 into nearby components.

Diffusion is one such mechanism. Diffusion is driven by concentrated gradients. When there is a drug concentration in the fluid inside the catheter extension set 50 and essentially no drug concentration in the fluid inside the catheter 900 itself, diffusion will cause drug/substance to travel into or toward the catheter region. Diffusion can exist even in the absence of bulk fluid motion such as convection. However, diffusion alone might not be sufficient to cause drug to disperse significant distances such as a distance of many inches into a catheter 900 in a reasonable period of time.

Another possible mass transfer mechanism is any mechanism that involves convection, i.e., actual bulk motion of fluid.

Even in fluid that generally or macroscopically appears to be stagnant, there might be some amount of convection occurring on some size scale or time scale. For example, a CVC (central venous catheter) as used for dialysis has side holes near its tip, and as soon as the catheter is placed, the blood flows through side hole and washes out at least some of the catheter lock solution. There are a variety of local blood flow patterns that can exist near the tips of various types of catheters. This can cause catheter lock fluid that was next to the sidewall to move. Also, the blood and the catheter lock solution usually have different density, so if catheter lock liquid is less dense than blood and if the patient lies on the appropriate side, there can be a gradient of density carrying blood into catheter and displacing catheter lock solution. As yet another phenomenon, there is heartbeat, and changes in pressure in the vena cava, and there is motion by the patient during ordinary activities. Also, when dialysis is performed one of first steps is to draw blood. That blood is not always discarded; sometimes it is re-injected back into the patient. Similarly, when a health care professional injects a catheter with saline, this step does not eliminate all of the catheter lock substance and some of the catheter lock substance may travel further along the catheter extension or catheter providing beneficial effect. So, there are a number of forms of convection (bulk fluid motion) that can act to spread the released substance, in addition to ordinary diffusion. It is possible that these combinations of mechanisms may spread the substance faster than ordinary diffusion alone would spread it. Even though the beneficial substance is only being eluted within the catheter extension tube 200, this still could protect a suitable length of the catheter 900 itself or catheter extension tube 200 itself or catheter extension set 1000 against growth of biofilm and microorganisms. In an embodiment, it would further be possible for the internal surface of a tube 200 to be textured rather than smooth. This might present an increased surface area to the liquid present in the lumen, thereby increasing the elution of drug to the fluid.

Experimental Results and Quantitative Methods for Assessing/Measuring Drug Delivery and Catheter Protection from Infection

Experiments were conducted for the release of drug from the described material, for the geometry in which tubing is made of the described material and drug is released to fluid (phosphate buffered saline) contained within the tubing. The experimental arrangement is shown in FIGS. 8A-8C. The fluid was used to fill the tubing and then remained stagnant inside the tubing for a specified interval of time, and then the fluid was removed from the tubing and the drug concentration in the fluid was measured. Fresh fluid was then used to refill the tubing for the next measurement, and the process was repeated approximately daily. The fluid was stagnant inside the tube at room temperature (25 Celsius). The length of exposed drug releasing polymer tubing was 15 mm. The tubing had a 5 mm Inside Diameter, with 1 mm wall thickness. In contrast, data in the parent patent application U.S. Ser. No. 17/986,604 was taken in regard to small coupons cut from a flat sheet, with the coupons being immersed in a bath that was agitated and was temperature controlled at 37 Celsius. For the current experiments, the substance used was Blend 9 as described in the parent patent application, which contains 60% EVA, 10% rifampin, 10% minocycline, 10% PEG and 10% polycaprolactone, as described in the parent patent application. In the present data, one set of results with 69 data points in 69 days is for the situation in which the upper end of the tubing was exposed to atmospheric air, which is believed to have resulted in some evaporation of the test liquid. In the set of results with only 51 data points, in 51 days, the upper end of the tubing was filled with a barrier fluid of saline or phosphate buffered saline, so that any evaporation that might occur was not from the fluid that would later be measured for its drug concentration to determine drug release during an interval. In both sets of data, the top of the tube was wrapped in parafilm (a thin sheet of paraffin wax) to prevent evaporation, but it is believed that some evaporation occurred despite the presence of the parafilm. The reason for the second set of data (51 data points in 51 days) with the extra fluid in the extra tubing is so that whatever evaporation did occur occurred from the fluid in the extra tubing, rather than from the fluid that interacted with the drug-eluting polymer.

Construct 1 refers to an experimental setup wherein a 15 mm length of antibiotic loaded tube is connected to representative catheter tubing. The antibiotic loaded tubing is 3.175 mm Inside Diameter and 60 cm in length. The top of the antibiotic tubing is sealed with parafilm. The total volume of the construct is 3.5 mL. This is supported in a vertical orientation by a laboratory ring stand. Construct 1 was run for an experimental duration of 69 days. Construct 2 refers to the same experimental arrangement as construct 1, with an additional 7 cm of non-antibiotic tubing at the upper end of the antibiotic loaded tube to mitigate evaporation that may occur due to imperfect parafilm seal. Instead of evaporating from the antibiotic loaded section of the tube, evaporation would occur from the saline in the non antibiotic loaded section, thereby allowing for more consistent release. Construct 2 was run for an experimental duration of 51 days. Construct 2 had a volume of 3.65 mL.

In FIGS. 9A-9D, on the right hand vertical axis, the concentration is expressed as a multiple of the Minimum Inhibitory Concentration, whose value is 0.65 micrograms/mL for minocycline. The value for rifampin is 0.095 micrograms/mL. This MIC is for Staphylococcus aureus. A therapeutic concentration of a drug in a fluid can be considered to be a defined multiple of the Minimum Inhibitory Concentration for growth of a microorganism of interest, or the Minimum Inhibitory Concentration for biofilm. Such multiple can, for example, be 10 or 20 or more, as desired, for inhibition and for preventing the development of resistant strains.

In the experiments illustrated in FIGS. 8A-8C, the drug-eluting polymer region of tubing was a 15 mm long segment of tubing that was near the top of the illustrated apparatus and was held by a laboratory clamp. Proceeding generally vertically downward from that region was a length of clear laboratory tubing tube filled with clear stagnant phosphate buffered saline. Rifampin naturally has a red-orange color. Thus, a visual observation is possible of how rifampin elutes into the liquid in the drug-eluting tubing segment and subsequently how the rifampin travels beyond the drug-eluting tubing segment into the clear fluid-filled segment by diffusion or by any other mass transfer mechanism. The dimensional extent of the mass transfer, and approximately the concentration of transferred drug, can be visually observed by the color of liquid at locations in the clear tubing beyond the drug-eluting tubing region. This is shown in FIG. 10. It can be seen that the front of the colored fluid, indicating presence of antibiotic, has advanced downward by a distance of about 4 cm.

With this observation, another experiment was performed, using an arrangement shown in FIG. 11. A 1.5875 mm Inside Diameter tube of ordinary clear laboratory tubing (not containing antibiotic) was filled with saline and was capped with a disc of the drug eluting polymer. The portion of the disc that was in contact with the saline and was considered to contribute antibiotic to the fluid in the interior of the tubing contained about 117 micrograms of rifampicin. This is calculated based on the overall weight of the coupon, which was 4 mg, 10% of which is rifampicin, and the contacted volume of the polymer was 0.79 mm\hat{ }3. Within the fluid, the front, as indicated by color of liquid, was observed visually to diffuse a distance of 3 centimeters in 30 minutes. So, the velocity of diffusion front, due to diffusion resulting from release of drug from the drug releasing polymer, per cross-sectional area and per microgram at the beginning of the catheter, is found to be 0.017 mm/min microgram. Over the course of 3 days (which is a typical duration of time at which to replace saline lock fluid in a catheter), if it is desired to protect a 15 cm long catheter, it will be necessary to provide 2.1 micrograms of drug per mm\hat{ }2 of cross-sectional area. More generally, for representative conditions, it is possible to achieve velocities of the diffusion front in the range of from 0.1 to 1 mm/min microgram.

FIGS. 13A-B present the same data presented in FIGS. 9A and 9C, and is normalized instead by the length of representative catheter tubing it should protect. This gives an idea of the total distance traveled for each day by the moving front for each data point.

It can be understood that for a given arrangement, the velocity of the front decreases according to the cross-sectional area of the tube, because a given amount of drug will be spread over that area and will diffuse less rapidly if the area is larger. Also for a given arrangement, the velocity of the front is related to the amount of drug present, as represented by the initial concentration of drug in the wall of the tube. Accordingly, both of these quantities are used together to normalize data presented here. In an embodiment, an achieved velocity of the front by diffusion may be in the range of from 0.1 to 1 mm/min microgram.

### Further Comments

In regard to beneficial substances that are released from the polymeric substance, generally any such beneficial substance is contemplated. Such beneficial substance could be an antibiotic, or an antimicrobial, antiseptic (one without cellular toxicity), drug, or generally anything that destroys or suppresses the growth of microorganisms or biofilm.

It can be pointed out that the described invention has a number of advantages.

In comparison to the known product ClearGuard, which uses chlorhexidine (an antiseptic that cannot be allowed to mix with bodily fluids), embodiments of the invention use antibiotics that are acceptable for exposure to the patient's body, and the antibiotics may released to the same places in the catheter system as ClearGuard releases, but indeed also may be released to more places including places that are in communication with bodily fluids of the patient.

Embodiments of the catheter extender-lengthener may be usable with existing catheter assemblies or catheter extension sets without requiring any geometric or manufacturing modification to the existing catheter assemblies or catheter extension sets. At worst, there could be a need for appropriate adapter fittings. Embodiments of the invention are relevant to a variety of medical treatments that involve catheters, including hemodialysis (using catheters in the absence of a fistula), peritoneal dialysis, cancer, chronic catheter for drainage, and heart conditions. For example, some heart medications need to be delivered intravenously near the heart. Embodiments of the invention can provide significant benefit for patients who have a catheter for any reason while also being immunosuppressed. Infection such as catheter-related blood stream infection (CRBSI) can be a significant complication for patients recovering from surgery or other therapy. Embodiments of the invention can be used with a Central Venous Catheter or with any other kind of catheter. It can be noted that in the US approximately 5,000,000 Central Venous Catheters are used each year, along with about 100,000 hemodialysis catheters.

Embodiments of the catheter extender-lengthener 50 described herein, as long as appropriate connector designs are provided, can be used with the technology of existing catheters without requiring any change to existing catheter designs. Furthermore, embodiments of the catheter extender-lengthener 50 described herein could be used in combination with a catheter that already is implanted in a patient without requiring changing out or removal or replacement of the catheter. Use of embodiments of the catheter extender-lengthener 50 would simply involve attaching the catheter extender-lengthener 50 of an embodiment of the invention to an existing catheter or catheter extension set 1000. Then, whatever would ordinarily be attached next, whether it be a cap or a medical device, could be attached to the end of the catheter extender-lengthener 50. For example, every peritoneal dialysis catheter has a catheter extension set or a transfer set, and embodiments of the invention could be used with that set. Typically, various peritoneal dialysis product supply companies have their own proprietary designs of connector. If necessary, in embodiments, the catheter extender-lengthener 50 could be supplied together with adapters that convert between proprietary connectors and more universal connectors. The catheter extender-lengthener 50 could be supplied together with the peritoneal dialysis catheter set.

There are many possible variations in the use of embodiments of the invention, either alone or in combination. For example, it is possible that the tubing of the catheter extension set 1000 could contain antibiotic-eluting polymer as described herein. In the same catheter assembly 2000, the catheter 900 itself might contain no antibiotic or, alternatively, might contain a different concentration of antibiotic, such as a smaller concentration of antibiotic. The tubing of the catheter 900 and the tubing of the catheter extension set 1000 could be made by different processes or could contain different polymer material or different antibiotics. For example, the catheter 900 could have its antibiotic added by solvent deposition (infusion), as described by Darouiche, while the tubing could be tubing that is as described herein and in the ancestor patent applications. If antibiotic is contained in the catheter 900 itself, that antibiotic could be the same antibiotic as is contained in the catheter extension set 1000, or in a catheter extender-lengthener 50, or could be a different antibiotic. Similarly, the concentrations or amounts of antibiotic in any of these items could vary, as could the materials of construction. The described substance-releasing tubing could be used in any catheter accessory device.

It would be possible to use a catheter assembly 2000 that contains antibiotic in the tubing of the catheter extension set 1000, and to use that catheter assembly 2000 for a period of time, and then when the elution of antibiotic from the tubing of the catheter extension set 1000 becomes undesirably small, it would be possible to add a catheter extender-lengthener 50 that elutes antibiotic. This could prolong the life of the implanted catheter 900 and avoid or postpone the need for replacement of that catheter 900. When the drug release from the catheter extender-lengthener 50 becomes undesirably small, it would be possible to replace that catheter extender-lengthener 50 with a different one. Yet another possibility would be to use an antibiotic-eluting cap 2800 as described herein. Such a cap 2800 could be used on the end of the catheter extender-lengthener 50, or could be used on the end of the catheter extension set 1000. Description herein about catheters, catheter extension sets, catheter assemblies and catheter extender-lengtheners apply equally well to projections 2850 from a cap 2800.

With embodiments of the invention, replacing the catheter extender-lengthener is easy, compared to replacing an entire catheter in a patient. In general, an embodiment of the invention can be connected onto a catheter system anywhere that fittings are compatible.

In embodiments, the interval at which the catheter extender-lengthener needs to be replaced might be different from the interval at which the catheter (or catheter extension set) itself needs to be replaced. Different embodiments of the invention could be made containing different antibiotics, and change of antibiotic or its dosage could be made simply by swapping out the catheter extender-lengthener. Change of antibiotic or its dosage could be made simply by swapping out an antibiotic-containing cap 2800.

Embodiments of the invention could be used with any polymer that releases a drug or desired substance. This of course includes the ethylene vinyl acetate family, but also includes silicones and polyurethanes, polyvinylpyrrolidone, elastomers in general, and generally also includes any other material capable of releasing a drug or desired substance.

In embodiments of the invention, it is possible that the polymer and the drug(s) and possibly other substances can co-exist in a material by mixing at a molecular level, or in the form of particulate blended with surrounding material, or in the form of a co-continuous network, or in discrete phases, or in any other configuration. It is intended that terms such as impregnated, blended, mixed, deposited, infused, etc. encompass all of these meanings. It also is possible that there could be a sleeve of one material inside another material. Although there is disclosed tubing made of the described material, it also is possible that in embodiments of the invention, other more rigid parts of a catheter extension set or catheter assembly or catheter extender-lengthener could be made of a material that comprises polymer and antibiotic and is capable of eluting the antibiotic.

If patients use a CVC for dialysis or for infusion, or for other purposes, the CVC could be an embodiment of the invention.

The terms catheter extender-lengthener and transfer set are both intended to be within the scope of the invention.

As used herein, various terms such as beneficial material, antibiotic, antiseptic, antimicrobial, antibacterial, drug, etc. are all intended to be within the scope of the invention. Any known antibiotic could be used.

All of these comments are applicable for any type of body access catheter or catheter extension set or catheter assembly or catheter extender-lengthener or tubing.

All references cited herein are incorporated by reference herein.

1. A catheter extender-lengthener, comprising:
a first connector, said first connector being suitable to engage with a corresponding connector of a catheter or a catheter extension set;
at least one catheter extender-lengthener tube proceeding from said first connector, said catheter extender-lengthener tube having a catheter extender-lengthener tube wall; and
a second connector proceeding from said catheter extender-lengthener tube,
wherein said catheter extender-lengthener tube wall comprises a polymer and at least one antibiotic.

2. The catheter extender-lengthener of claim 1, wherein said at least one antibiotic is present in said catheter extender-lengthener tube wall at a concentration of at least 2% by weight.

3. The catheter extender-lengthener of claim 1, wherein said polymer and said at least one antibiotic exist together in said material by mixing at a molecular level, or in the form of particulate blended with surrounding material, or in the form of a co-continuous network, or in discrete phases.

4. (canceled)

5. The catheter extender-lengthener of claim 1, wherein said catheter extender-lengthener tube wall is capable of releasing a therapeutic amount of said at least one antibiotic, to a fluid contained within said catheter extender-lengthener tube, for at least 30 days.

6. (canceled)

7. The catheter extender-lengthener of claim 1, wherein said at least one antibiotic is selected from the group consisting of: minocycline; other members of the tetracyclines class of antibiotics; rifampicin; other antimycobacterial drugs; ciprofloxacin; other fluoroquinolones; vancomycin; other glycopeptides; cephalosporins; penicillins; gentamicin; other aminoglycosides; sulfonamides; macrolides; carbapenems; linezolid; other oxazolidinones; daptomycin; other lipopeptides; and immunosuppressants.

8. The catheter extender-lengthener of claim 1, wherein said polymer is selected from the group consisting of: ethylene vinyl acetate; silicone; polyurethane; polyvinylpyrrolidone; polytetrafluoroethylene; polyvinyl chloride; rubber; latex rubber and elastomers.

9. The catheter extender-lengthener of claim 1, wherein said polymer comprises ethylene vinyl acetate and a release modifying material comprising polyethylene glycol or polycaprolactone or both.

10. (canceled)

11. The catheter extender-lengthener of claim 1, wherein said catheter extender-lengthener tube wall comprises an inner layer and an outer layer, wherein said inner layer comprises said polymer and said at least one antibiotic and said outer layer does not contain any of said at least one antibiotic, or wherein said catheter extender-lengthener tube comprises, for a first portion of its length, a first material that comprises said polymer and said at least one antibiotic, and comprises, for a second portion of its length, a second material that does not comprise any of said at least one antibiotic.

12.-14. (canceled)

15. The catheter extender-lengthener of claim 1, wherein said catheter extender-lengthener tube comprises 60% (w/w) ethylene vinyl acetate, 10% polyethylene glycol, 10% polycprolactone, 10% minocycline and 10% rifampin.

16. (canceled)

17. The catheter extender-lengthener of claim 1, in combination with a cap that is mateable to said second connector, said cap being capable of releasing an antimicrobial sub stance.

18. (canceled)

19. A catheter extender-lengthener for use with a catheter, said catheter extender-lengthener comprising:
a first connector, said first connector being suitable to engage with a corresponding connector of a catheter or a catheter extension set;
a catheter extender-lengthener tube proceeding from said first connector, said catheter extender-lengthener tube having a catheter extender-lengthener tube wall containing at least one antibiotic substance that is safe for exposure to a patient's bodily fluids; and
a second connector proceeding from said catheter extender-lengthener tube;
said first connector, said catheter extender-lengthener tube and said second connector defining a lumen extending therethrough,
further comprising a clamp disposed on said catheter extender-lengthener tube having at least one permitted location on said catheter extender-lengthener tube,
wherein when said clamp is in a permitted location on said catheter extender-lengthener tube, fluid inside said lumen on one side of said clamp is exposed to said polymer and said at least one antibiotic substance, and fluid inside said lumen on an opposite side of said clamp is exposed to said polymer and said at least one antibiotic substance.

20. (canceled)

21. The catheter extender-lengthener of claim 19, wherein said at least one antibiotic substance comprises minocycline and rifampin.

22. The catheter extender-lengthener of claim 19, further comprising a cap mateable with said second connector, wherein said cap comprises said polymer and said at least one antibiotic substance, wherein said polymer and said at least one antibiotic substance are in a projection that is attached to or integral with said cap, and wherein when said cap is mated with said second connector, said projection extends through said clamp.

23.-25. (canceled)

26. The catheter extender-lengthener of claim 24, wherein said catheter extender-lengthener tube has texturing on an internal surface thereof, suitable to increase its internal surface area compared to that of a smooth surface.

27. A catheter assembly of a catheter and a catheter extension set, said catheter assembly comprising:
said catheter, said catheter being suitable to extend into a patient's body;
a junction between said catheter and said catheter extension set; and
said catheter extension set, said catheter extension set comprising:
at least one catheter extension tube proceeding from said junction, said catheter extension tube having a catheter extension tube wall; and
an end connector proceeding from said catheter extension tube,
wherein said catheter, said junction, said catheter extension tube and said end connector define a lumen extending therethrough,
wherein said catheter extension tube wall comprises a polymer and at least one antibiotic or combination of antibiotics, and
wherein said catheter extension tube wall has a composition different from a composition of said catheter.

28.-30. (canceled)

31. The catheter assembly of claim 27, wherein said catheter extension tube wall is capable of releasing a therapeutic amount of said at least one antibiotic, to a fluid contained within said catheter extension tube, for at least 30 days.

32.-34. (canceled)

35. The catheter assembly of claim 27, wherein said polymer comprises ethylene vinyl acetate and a release modifying material comprising polyethylene glycol or polycaprolactone or both.

36. The catheter assembly of claim 27, wherein said catheter assembly comprises more than one of said catheter extension tube, each of said catheter extension tubes having a respective end connector.

37. The catheter assembly of claim 27, wherein said catheter extension tube wall comprises an inner layer and an outer layer, wherein said inner layer comprises said polymer and said at least one antibiotic and said outer layer does not contain any of said at least one antibiotic, or wherein said catheter extension tube comprises, for a first portion of its length, a first material that comprises said polymer and said at least one antibiotic, and comprises, for a second portion of its length, a second material that does not comprise any of said at least one antibiotic.

38.-39. (canceled)

40. The catheter assembly of claim 27, in combination with a cap that is mateable to said end connector, said cap being capable of releasing an antimicrobial sub stance.

41. (canceled)

42. The catheter assembly of claim 27, wherein said catheter either does not contain any of said at least one antibiotic or contains a smaller concentration of said at least one antibiotic than is present in said catheter extension tube.

43. (canceled)

44. A cap for connecting onto a connector, said cap comprising:
an engagement means for engaging with said connector and forming a fluid-tight seal with said connector; and
a projection connected to said cap, wherein said projection comprises a polymer and at least one antibiotic that is safe for exposure to a patient's bodily fluids.

45. The cap of claim 44, wherein said connector is an end connector of a catheter assembly or is an end connector of a catheter extender-lengthener.

46. (canceled)

47. The cap of claim 44, in combination with tubing, said tubing having a clamp disposed thereon, wherein said projection is suitable to extend into said tubing farther than said clamp.

48.-50. (canceled)

51. The cap of claim 44, wherein said projection is capable of releasing a therapeutic amount of said at least one antibiotic, to a fluid contained within said tubing, for at least 30 days.

52. (canceled)

53. The cap of claim 44, wherein said at least one antibiotic comprises minocycline and rifampin and said polymer comprises ethylene vinyl acetate and polyethylene glycol and polycaprolactone.

54. The cap of claim 44, wherein said polymer comprises silicone or polyurethane or polyvinylpyrrolidone or an elastomer, or comprises ethylene vinyl acetate and polyethylene glycol and polycaprolactone.

55.-58. (canceled)
